SEQUENCE LISTING (171 sequences)

SEQ ID NO: 1 - DNA, 17 bp, synthetic construct
  source 1..17: mol_type = other DNA
tccgtgtggcggcctag

SEQ ID NO: 2 - DNA, 21 bp, synthetic construct
  source 1..21: mol_type = other DNA
caaccttagagacaggtacag

SEQ ID NO: 3 - DNA, 21 bp, synthetic construct
  source 1..21: mol_type = other DNA
aggcaggtacacagtggcctc

SEQ ID NO: 4 - DNA, 19 bp, synthetic construct
  source 1..19: mol_type = other DNA
cagacgtcacagactccag

SEQ ID NO: 5 - DNA, 19 bp, synthetic construct
  source 1..19: mol_type = other DNA
cacggattgttgtccccgc

SEQ ID NO: 6 - DNA, 21 bp, synthetic construct
  source 1..21: mol_type = other DNA
acaccaagtaccccaacaacc

SEQ ID NO: 7 - DNA, 18 bp, synthetic construct
  source 1..18: mol_type = other DNA
ggctcgcagacgtcctac

SEQ ID NO: 8 - DNA, 21 bp, synthetic construct
  source 1..21: mol_type = other DNA
ctgaggaagacgatttagctg

SEQ ID NO: 9 - DNA, 19 bp, synthetic construct
  source 1..19: mol_type = other DNA
cagggcgtttgcgagggcc

SEQ ID NO: 10 - DNA, 21 bp, synthetic construct
  source 1..21: mol_type = other DNA
ctggtaaattaggagaggctg

SEQ ID NO: 11 - DNA, 20 bp, synthetic construct
  source 1..20: mol_type = other DNA
aatagaggcagtagttcctg

SEQ ID NO: 12 - DNA, 23 bp, synthetic construct
  source 1..23: mol_type = other DNA
cccgatgacctgtacattaaagg

SEQ ID NO: 13 - DNA, 23 bp, synthetic construct
  source 1..23: mol_type = other DNA
tagcaaccaaatatccactactg

SEQ ID NO: 14 - DNA, 18 bp, synthetic construct
  source 1..18: mol_type = other DNA
cgctgcttgtggtggtcg

SEQ ID NO: 15 - DNA, 23 bp, synthetic construct
  source 1..23: mol_type = other DNA
ctgctgacatacagacagacaac

SEQ ID NO: 16 - DNA, 18 bp, synthetic construct
  source 1..18: mol_type = other DNA
cctgcccactgacctgta

SEQ ID NO: 17 - DNA, 18 bp, synthetic construct
  source 1..18: mol_type = other DNA
gtaactgcccggcatttg

SEQ ID NO: 18 - DNA, 20 bp, synthetic construct
  source 1..20: mol_type = other DNA
ctgtcactgcctggggtacc

SEQ ID NO: 19 - DNA, 19 bp, synthetic construct
  source 1..19: mol_type = other DNA
gggtgaggaactaccagcc

SEQ ID NO: 20 - DNA, 18 bp, synthetic construct
  source 1..18: mol_type = other DNA
caggtgtcaccctgtcgc

SEQ ID NO: 21 - DNA, 25 bp, synthetic construct
  source 1..25: mol_type = other DNA
cccgaggcacctttaagatataaac

SEQ ID NO: 22 - DNA, 23 bp, synthetic construct
  source 1..23: mol_type = other DNA
ccttctgctactactaaaaaacg

SEQ ID NO: 23 - DNA, 18 bp, synthetic construct
  source 1..18: mol_type = other DNA
gtttggctgatggggtag

SEQ ID NO: 24 - DNA, 20 bp, synthetic construct
  source 1..20: mol_type = other DNA
cggcccccagttcctctacc

SEQ ID NO: 25 - DNA, 18 bp, synthetic construct
  source 1..18: mol_type = other DNA
ggcgtacaacgcaagccc

SEQ ID NO: 26 - DNA, 21 bp, synthetic construct
  source 1..21: mol_type = other DNA
ctgaagaggaagaggaagagg

SEQ ID NO: 27 - DNA, 19 bp, synthetic construct
  source 1..19: mol_type = other DNA
ccaggccttaaacgcccgg

SEQ ID NO: 28 - DNA, 19 bp, synthetic construct
  source 1..19: mol_type = other DNA
caatccccttaccacctgc

SEQ ID NO: 29 - DNA, 20 bp, synthetic construct
  source 1..20: mol_type = other DNA
cgctgtcgttttcgctgttg

SEQ ID NO: 30 - DNA, 19 bp, synthetic construct
  source 1..19: mol_type = other DNA
ctgggcttctttggcgccc

SEQ ID NO: 31 - DNA, 19 bp, synthetic construct
  source 1..19: mol_type = other DNA
agaggccagtgccattcgt

SEQ ID NO: 32 - DNA, 21 bp, synthetic construct
  source 1..21: mol_type = other DNA
gtttctctgcgtcgttggagt

SEQ ID NO: 33 - DNA, 22 bp, synthetic construct
  source 1..22: mol_type = other DNA
tcctgtcgtgctcggttgcagc

SEQ ID NO: 34 - DNA, 21 bp, synthetic construct
  source 1..21: mol_type = other DNA
tttttaatagggccggtactg

SEQ ID NO: 35 - DNA, 19 bp, synthetic construct
  source 1..19: mol_type = other DNA
acccccttttaccaacagg

SEQ ID NO: 36 - DNA, 19 bp, synthetic construct
  source 1..19: mol_type = other DNA
atcaggcacaggttccccc

SEQ ID NO: 37 - DNA, 19 bp, synthetic construct
  source 1..19: mol_type = other DNA
ctggtatacagcgacgtcc

SEQ ID NO: 38 - DNA, 21 bp, synthetic construct
  source 1..21: mol_type = other DNA
aggaggaagatgcagataagg

SEQ ID NO: 39 - DNA, 20 bp, synthetic construct
  source 1..20: mol_type = other DNA
ctggacgtttggtgcctaac

SEQ ID NO: 40 - DNA, 17 bp, synthetic construct
  source 1..17: mol_type = other DNA
agcggggggctgccccc

SEQ ID NO: 41 - DNA, 17 bp, synthetic construct
  source 1..17: mol_type = other DNA
tgggcgccgccgcagtt

SEQ ID NO: 42 - DNA, 20 bp, synthetic construct
  source 1..20: mol_type = other DNA
cagcggcgcgttttcgagac

SEQ ID NO: 43 - DNA, 19 bp, synthetic construct
  source 1..19: mol_type = other DNA
gccctaggaaacgtgttgc

SEQ ID NO: 44 - DNA, 20 bp, synthetic construct
  source 1..20: mol_type = other DNA
agcagtacgtttccgtttgg

SEQ ID NO: 45 - DNA, 20 bp, synthetic construct
  source 1..20: mol_type = other DNA
cttctactgctactaggccc

SEQ ID NO: 46 - DNA, 21 bp, synthetic construct
  source 1..21: mol_type = other DNA
cttcacaggccattacatgtc

SEQ ID NO: 47 - DNA, 23 bp, synthetic construct
  source 1..23: mol_type = other DNA
cattttagcatatggatcctcag

SEQ ID NO: 48 - DNA, 20 bp, synthetic construct
  source 1..20: mol_type = other DNA
ccttaggaggttgcggacgc

SEQ ID NO: 49 - DNA, 18 bp, synthetic construct
  source 1..18: mol_type = other DNA
gcaggttggggtccgtac

SEQ ID NO: 50 - DNA, 21 bp, synthetic construct
  source 1..21: mol_type = other DNA
gggctgaggtagatgtagtag

SEQ ID NO: 51 - DNA, 25 bp, synthetic construct
  source 1..25: mol_type = other DNA
cctgtatcctctaaaaaacgctctg

SEQ ID NO: 52 - DNA, 19 bp, synthetic construct
  source 1..19: mol_type = other DNA
ccagaaaacacctccctgg

SEQ ID NO: 53 - DNA, 21 bp, synthetic construct
  source 1..21: mol_type = other DNA
ggtgcagtcttataaccactg

SEQ ID NO: 54 - DNA, 22 bp, synthetic construct
  source 1..22: mol_type = other DNA
cggtgttgttacacgaagctcc

SEQ ID NO: 55 - DNA, 19 bp, synthetic construct
  source 1..19: mol_type = other DNA
caggatatagggcacgtcc

SEQ ID NO: 56 - DNA, 21 bp, synthetic construct
  source 1..21: mol_type = other DNA
ctggtgtagtggtagatgctg

SEQ ID NO: 57 - DNA, 22 bp, synthetic construct
  source 1..22: mol_type = other DNA
agcaggtaaacgtagtgcaccc

SEQ ID NO: 58 - DNA, 19 bp, synthetic construct
  source 1..19: mol_type = other DNA
gacagtgtcaactgtgggg

SEQ ID NO: 59 - DNA, 19 bp, synthetic construct
  source 1..19: mol_type = other DNA
gcaggacaactgacagccc

SEQ ID NO: 60 - DNA, 22 bp, synthetic construct
  source 1..22: mol_type = other DNA
cagtgcagctgcatgcacaaac

SEQ ID NO: 61 - DNA, 17 bp, synthetic construct
  source 1..17: mol_type = other DNA
actgcaggccgggttgc

SEQ ID NO: 62 - DNA, 21 bp, synthetic construct
  source 1..21: mol_type = other DNA
tttagctgtagacgcctttcg

SEQ ID NO: 63 - DNA, 23 bp, synthetic construct
  source 1..23: mol_type = other DNA
cctacagacagtttaggccttgc

SEQ ID NO: 64 - DNA, 21 bp, synthetic construct
  source 1..21: mol_type = other DNA
tagcttgtatgtcattccacg

SEQ ID NO: 65 - DNA, 19 bp, synthetic construct
  source 1..19: mol_type = other DNA
gggcaggtatacctttgcc

SEQ ID NO: 66 - DNA, 20 bp, synthetic construct
  source 1..20: mol_type = other DNA
cagtaggtcgccacatctgc

SEQ ID NO: 67 - DNA, 18 bp, synthetic construct
  source 1..18: mol_type = other DNA
tgtgtcccgcaagcgtcc

SEQ ID NO: 68 - DNA, 20 bp, synthetic construct
  source 1..20: mol_type = other DNA
ggaacgtttggaccgtttac

SEQ ID NO: 69 - DNA, 21 bp, synthetic construct
  source 1..21: mol_type = other DNA
tgtagagggggcggtagaggg

SEQ ID NO: 70 - DNA, 21 bp, synthetic construct
  source 1..21: mol_type = other DNA
gcacaccacctataggtgaac

SEQ ID NO: 71 - skipped

SEQ ID NO: 72 - DNA, 23 bp, synthetic construct
  source 1..23: mol_type = other DNA
tgggctaaaggcaacctatgtac

SEQ ID NO: 73 - DNA, 23 bp, synthetic construct
  source 1..23: mol_type = other DNA
ggggaatcagttatttgttactg

SEQ ID NO: 74 - DNA, 21 bp, synthetic construct
  source 1..21: mol_type = other DNA
ttgtagatggagactgagttg

SEQ ID NO: 75 - DNA, 26 bp, synthetic construct
  source 1..26: mol_type = other DNA
cgtagtacaaacatgacaatatgtgc

SEQ ID NO: 76 - DNA, 19 bp, synthetic construct
  source 1..19: mol_type = other DNA
cgcagggccacaataatgg

SEQ ID NO: 77 - DNA, 19 bp, synthetic construct
  source 1..19: mol_type = other DNA
cggaggggactgtgtagtg

SEQ ID NO: 78 - DNA, 22 bp, synthetic construct
  source 1..22: mol_type = other DNA
ctacccgtagtacaaacatgac

SEQ ID NO: 79 - DNA, 22 bp, synthetic construct
  source 1..22: mol_type = other DNA
ccaaagttttaagtacggaccg

SEQ ID NO: 80 - DNA, 20 bp, synthetic construct
  source 1..20: mol_type = other DNA
ctgtaataaggcgggagctg

SEQ ID NO: 81 - DNA, 22 bp, synthetic construct
  source 1..22: mol_type = other DNA
tgtccagcgtaccaacctgtac

SEQ ID NO: 82 - DNA, 18 bp, synthetic construct
  source 1..18: mol_type = other DNA
ggctttgtggcggcctag

SEQ ID NO: 83 - DNA, 19 bp, synthetic construct
  source 1..19: mol_type = other DNA
caactctggccacagaagg

SEQ ID NO: 84 - DNA, 22 bp, synthetic construct
  source 1..22: mol_type = other DNA
acagtacggtatatcttccacc

SEQ ID NO: 85 - DNA, 21 bp, synthetic construct
  source 1..21: mol_type = other DNA
cccaattggggaatattgggg

SEQ ID NO: 86 - DNA, 18 bp, synthetic construct
  source 1..18: mol_type = other DNA
gtccccaagggttacccg

SEQ ID NO: 87 - DNA, 20 bp, synthetic construct
  source 1..20: mol_type = other DNA
agggcactccgtgcaatgct

SEQ ID NO: 88 - DNA, 22 bp, synthetic construct
  source 1..22: mol_type = other DNA
tcagtttcctttgggacgaaag

SEQ ID NO: 89 - DNA, 19 bp, synthetic construct
  source 1..19: mol_type = other DNA
gatggtgcacgggtagtag

SEQ ID NO: 90 - DNA, 20 bp, synthetic construct
  source 1..20: mol_type = other DNA
caggccttaaagcaaagccc

SEQ ID NO: 91 - DNA, 20 bp, synthetic construct
  source 1..20: mol_type = other DNA
gagacaacacccagtactcc

SEQ ID NO: 92 - DNA, 19 bp, synthetic construct
  source 1..19: mol_type = other DNA
ctcctcgtggtctactgtc

SEQ ID NO: 93 - DNA, 21 bp, synthetic construct
  source 1..21: mol_type = other DNA
caaagtatacagactgcgccg

SEQ ID NO: 94 - DNA, 21 bp, synthetic construct
  source 1..21: mol_type = other DNA
gtggaattgcagttgctattc

SEQ ID NO: 95 - DNA, 21 bp, synthetic construct
  source 1..21: mol_type = other DNA
catgttaccctacagttgcag

SEQ ID NO: 96 - DNA, 21 bp, synthetic construct
  source 1..21: mol_type = other DNA
cttccctgtccactgtccagc

SEQ ID NO: 97 - DNA, 18 bp, synthetic construct
  source 1..18: mol_type = other DNA
gcaactgggcactaggtc

SEQ ID NO: 98 - DNA, 19 bp, synthetic construct
  source 1..19: mol_type = other DNA
gtggaggtaggagcagatc

SEQ ID NO: 99 - DNA, 21 bp, synthetic construct
  source 1..21: mol_type = other DNA
ttagaggtagctacagcaggc

SEQ ID NO: 100 - DNA, 21 bp, synthetic construct
  source 1..21: mol_type = other DNA
gaatttgactcctccagagag

SEQ ID NO: 101 - DNA, 20 bp, synthetic construct
  source 1..20: mol_type = other DNA
cggtattgtctgtgtcgctg

SEQ ID NO: 102 - DNA, 22 bp, synthetic construct
  source 1..22: mol_type = other DNA
cccacgcaaagtgtgtcacaac

SEQ ID NO: 103 - DNA, 21 bp, synthetic construct
  source 1..21: mol_type = other DNA
cattgtcccgtaaaagggttg

SEQ ID NO: 104 - DNA, 21 bp, synthetic construct
  source 1..21: mol_type = other DNA
ggagcgtttaacacgtttacg

SEQ ID NO: 105 - DNA, 18 bp, synthetic construct
  source 1..18: mol_type = other DNA
ccacatccaccgccccca

SEQ ID NO: 106 - DNA, 18 bp, synthetic construct
  source 1..18: mol_type = other DNA
caggccggcatacaacgg

SEQ ID NO: 107 - DNA, 21 bp, synthetic construct
  source 1..21: mol_type = other DNA
ggaagaggtagaagataaggg

SEQ ID NO: 108 - DNA, 22 bp, synthetic construct
  source 1..22: mol_type = other DNA
cgtttggtgcctagtttcggcc

SEQ ID NO: 109 - DNA, 18 bp, synthetic construct
  source 1..18: mol_type = other DNA
tgtccgccgacgacccac

SEQ ID NO: 110 - DNA, 23 bp, synthetic construct
  source 1..23: mol_type = other DNA
tttgacacacgtttacgtttgtg

SEQ ID NO: 111 - DNA, 20 bp, synthetic construct
  source 1..20: mol_type = other DNA
ataggcccccgcaaacgccc

SEQ ID NO: 112 - DNA, 18 bp, synthetic construct
  source 1..18: mol_type = other DNA
actgagccctctgagccc

SEQ ID NO: 113 - DNA, 19 bp, synthetic construct
  source 1..19: mol_type = other DNA
tctactgaggagtgggagg

SEQ ID NO: 114 - DNA, 19 bp, synthetic construct
  source 1..19: mol_type = other DNA
cgtgtccgtggacggtgtc

SEQ ID NO: 115 - DNA, 22 bp, synthetic construct
  source 1..22: mol_type = other DNA
gtatctgtgtcacgcaaacgtg

SEQ ID NO: 116 - DNA, 19 bp, synthetic construct
  source 1..19: mol_type = other DNA
gcgttttgtagcaggggag

SEQ ID NO: 117 - DNA, 18 bp, synthetic construct
  source 1..18: mol_type = other DNA
tgcccccagctctacccc

SEQ ID NO: 118 - DNA, 18 bp, synthetic construct
  source 1..18: mol_type = other DNA
ccacgcaaacgtgcagcg

SEQ ID NO: 119 - DNA, 21 bp, synthetic construct
  source 1..21: mol_type = other DNA
gacgcttaacacgttttggtg

SEQ ID NO: 120 - DNA, 19 bp, synthetic construct
  source 1..19: mol_type = other DNA
ccgcccctacctctacccc

SEQ ID NO: 121 - DNA, 19 bp, synthetic construct
  source 1..19: mol_type = other DNA
gtctaccagcgtgtcagtg

SEQ ID NO: 122 - DNA, 19 bp, synthetic construct
  source 1..19: mol_type = other DNA
gttgcctggatgcaaacgg

SEQ ID NO: 123 - DNA, 20 bp, synthetic construct
  source 1..20: mol_type = other DNA
ccactgtgacaacccagtcg

SEQ ID NO: 124 - DNA, 20 bp, synthetic construct
  source 1..20: mol_type = other DNA
gcgtgcacgtcctaagttac

SEQ ID NO: 125 - DNA, 20 bp, synthetic construct
  source 1..20: mol_type = other DNA
cttaggtgtggcacttgtgg

SEQ ID NO: 126 - DNA, 22 bp, synthetic construct
  source 1..22: mol_type = other DNA
cagatgcagaacgtttagaagc

SEQ ID NO: 127 - DNA, 19 bp, synthetic construct
  source 1..19: mol_type = other DNA
gtgaggtgctgcctacctc

SEQ ID NO: 128 - DNA, 23 bp, synthetic construct
  source 1..23: mol_type = other DNA
catagatgtagctggtaagagtg

SEQ ID NO: 129 - DNA, 23 bp, synthetic construct
  source 1..23: mol_type = other DNA
ccaggcacctctgctaatagtag

SEQ ID NO: 130 - DNA, 22 bp, synthetic construct
  source 1..22: mol_type = other DNA
taaatttttgttgcaggccagg

SEQ ID NO: 131 - DNA, 18 bp, synthetic construct
  source 1..18: mol_type = other DNA
cgaggaagtggatgcagc

SEQ ID NO: 132 - DNA, 20 bp, synthetic construct
  source 1..20: mol_type = other DNA
ccgcaggcgccctactatag

SEQ ID NO: 133 - DNA, 21 bp, synthetic construct
  source 1..21: mol_type = other DNA
gtccctggaccatcttaacaa

SEQ ID NO: 134 - DNA, 19 bp, synthetic construct
  source 1..19: mol_type = other DNA
ccacaactgaggtaccgtc

SEQ ID NO: 135 - DNA, 23 bp, synthetic construct
  source 1..23: mol_type = other DNA
ccactccacagtaacagtacagg

SEQ ID NO: 136 - DNA, 20 bp, synthetic construct
  source 1..20: mol_type = other DNA
cccaaggctagtgtatctgc

SEQ ID NO: 137 - DNA, 21 bp, synthetic construct
  source 1..21: mol_type = other DNA
gctggtgaagaagaggaagag

SEQ ID NO: 138 - DNA, 21 bp, synthetic construct
  source 1..21: mol_type = other DNA
ctaaaaagcgggcggctccta

SEQ ID NO: 139 - DNA, 22 bp, synthetic construct
  source 1..22: mol_type = other DNA
aacagacacagacatcagtaac

SEQ ID NO: 140 - DNA, 20 bp, synthetic construct
  source 1..20: mol_type = other DNA
caccacagtggttgtgtgtg

SEQ ID NO: 141 - DNA, 21 bp, synthetic construct
  source 1..21: mol_type = other DNA
cgccaacagtagaagtscacg

SEQ ID NO: 142 - DNA, 19 bp, synthetic construct
  source 1..19: mol_type = other DNA
gggaacctgtgcctgatac

SEQ ID NO: 143 - DNA, 24 bp, synthetic construct
  source 1..24: mol_type = other DNA
ggtgttaacatatatactactccc

SEQ ID NO: 144 - DNA, 22 bp, synthetic construct
  source 1..22: mol_type = other DNA
agggtagtggaaatcgcacgtc

SEQ ID NO: 145 - DNA, 19 bp, synthetic construct
  source 1..19: mol_type = other DNA
gctggtaaaactggcgacg

SEQ ID NO: 146 - DNA, 23 bp, synthetic construct
  source 1..23: mol_type = other DNA
caattttggacctggtattagag

SEQ ID NO: 147 - DNA, 23 bp, synthetic construct
  source 1..23: mol_type = other DNA
cagcaatatacatatcggaaggc

SEQ ID NO: 148 - DNA, 19 bp, synthetic construct
  source 1..19: mol_type = other DNA
gctcctcctattggggaac

SEQ ID NO: 149 - DNA, 19 bp, synthetic construct
  source 1..19: mol_type = other DNA
accctgaggtacaggtacg

SEQ ID NO: 150 - DNA, 20 bp, synthetic construct
  source 1..20: mol_type = other DNA
tgggccacaggtaccacatg

SEQ ID NO: 151 - DNA, 19 bp, synthetic construct
  source 1..19: mol_type = other DNA
ccaggaggcacactagaag

SEQ ID NO: 152 - DNA, 23 bp, synthetic construct
  source 1..23: mol_type = other DNA
ggaggtgtatgtttttgacaagc

SEQ ID NO: 153 - DNA, 24 bp, synthetic construct
  source 1..24: mol_type = other DNA
cttataggtttgtaacatcccagg

SEQ ID NO: 154 - DNA, 19 bp, synthetic construct
  source 1..19: mol_type = other DNA
cacagacgactatccagcg

SEQ ID NO: 155 - DNA, 18 bp, synthetic construct
  source 1..18: mol_type = other DNA
ggtgtggcaggggtttcc

SEQ ID NO: 156 - DNA, 20 bp, synthetic construct
  source 1..20: mol_type = other DNA
ccaagatcagagccagacac

SEQ ID NO: 157 - DNA, 19 bp, synthetic construct
  source 1..19: mol_type = other DNA
cacccggtcttttgctgtg

SEQ ID NO: 158 - DNA, 19 bp, synthetic construct
  source 1..19: mol_type = other DNA
gcttagcagaggtagtggg

SEQ ID NO: 159 - DNA, 20 bp, synthetic construct
  source 1..20: mol_type = other DNA
ccgtaagcgtcctgcgtctg

SEQ ID NO: 160 - DNA, 21 bp, synthetic construct
  source 1..21: mol_type = other DNA
gtgaaacagtacctgcagacc

SEQ ID NO: 161 - DNA, 21 bp, synthetic construct
  source 1..21: mol_type = other DNA
catagagccactaggagtagg

SEQ ID NO: 162 - DNA, 22 bp, synthetic construct
  source 1..22: mol_type = other DNA
agggtaccactggcacattgcc

SEQ ID NO: 163 - DNA, 19 bp, synthetic construct
  source 1..19: mol_type = other DNA
caccaagcgagtgcgactc

SEQ ID NO: 164 - DNA, 21 bp, synthetic construct
  source 1..21: mol_type = other DNA
gtcagatgtagagtagacccc

SEQ ID NO: 165 - DNA, 22 bp, synthetic construct
  source 1..22: mol_type = other DNA
ctgtcaacactgtccacggcac

SEQ ID NO: 166 - DNA, 19 bp, synthetic construct
  source 1..19: mol_type = other DNA
ggggaggatgtacctacag

SEQ ID NO: 167 - DNA, 22 bp, synthetic construct
  source 1..22: mol_type = other DNA
ggagtaggaaaaaaagctgagg

SEQ ID NO: 168 - DNA, 22 bp, synthetic construct
  source 1..22: mol_type = other DNA
tgcagaagtgtttgcagatccc

SEQ ID NO: 169 - DNA, 21 bp, synthetic construct
  source 1..21: mol_type = other DNA
ctagggttggccaatctactc

SEQ ID NO: 170 - DNA, 21 bp, synthetic construct
  source 1..21: mol_type = other DNA
ctgacttttatgcccagccct

SEQ ID NO: 171 - DNA, 22 bp, synthetic construct
  source 1..22: mol_type = other DNA
caggagcagggagggcaggagc

(19) United States
(12) Patent Application Publication
(10) Pub. No.: US 2026/0275432 A1
(43) Pub. Date: Sep. 17, 2026

(54) KIT FOR GENOTYPING OR DETECTION OF HPVs, METHOD AND SYSTEM USING SAID KIT

(71) Applicant: MedTimes Molecular Laboratory Limited, Kwai Chung (HK)

(72) Inventors: Chun Kin Chow, Kwai Chung (HK); Ka Ki Au, Kwai Chung (HK); Long Ling Ngan, Kwai Chung (HK); Nga Lam Poon, Kwai Chung (HK); Chu Hua Lai, Kwai Chung (HK); Wing Ki Chui, Kwai Chung (HK)

(21) Appl. No.: 19/567,577

(22) Filed: Mar. 16, 2026

Related U.S. Application Data
(60) Provisional application No. 63/771,909, filed on Mar. 14, 2025.

(51) Int. Cl. C12Q 1/70; C12Q 1/48; C12Q 1/6848; C12Q 1/686; C12Q 1/6876

(52) U.S. Cl. CPC C12Q 1/708; C12Q 1/48; C12Q 1/6848; C12Q 1/686; C12Q 1/6876; C12Y 207/07; C12Q 2600/112; C12Q 2600/166

(57) ABSTRACT

The present disclosure provides a kit, an in vitro method, and automated system for detecting and simultaneously genotyping Human Papillomavirus (HPV) genotypes in a biological sample. The method partitions the sample into a coordinated reaction set including eight physically discrete reaction pools. Each pool includes a distinct, specifically formulated subset of oligonucleotide triplets—including forward primers, reverse primers, and dual-labeled fluorescent DNA probes—engineered to amplify synergistic combinations of the viral L1, E4, and E6 gene loci. This multi-genic targeting strategy effectively circumvents false-negative results associated with viral episomal integration and L1 gene deletion.

### SEQUENCE LISTING

The Sequence Listing file entitled “sequencelisting.xml” having a size of 151009 bytes and a creation date of Mar. 16, 2026, that was filed with the patent application is incorporated herein by reference in its entirety.

### FIELD OF THE INVENTION

The present disclosure relates generally to the fields of molecular biology and in vitro diagnostics. More specifically, the invention relates to optimized compositions, reaction systems, and methods for the highly multiplexed amplification, detection, and precise clinical genotyping of Human Papillomavirus (HPV) target nucleic acids within a biological sample. In particular, the present application provides a robust molecular diagnostic architecture—utilizing targeted multiplex real-time polymerase chain reaction (qPCR) methodologies—that is capable of simultaneously detecting and uniquely differentiating a vast plurality of distinct HPV genotypes within a single, coordinated reaction set. By concurrently identifying and categorizing high-risk, intermediate-risk, and low-risk HPV strains, the disclosed methods and kits provide enhanced clinical utility for high-throughput epidemiological surveillance, diagnostic screening, and the precise oncogenic risk stratification of patients susceptible to HPV-induced dysplasias and carcinomas.

### BACKGROUND

Human Papillomaviruses (HPVs) are small, double-stranded DNA viruses that infect human epithelial cells. Persistent infections by specific high-risk HPV genotypes are unequivocally recognized as the primary causative agents for cervical carcinomas and other anogenital malignancies. While real-time Polymerase Chain Reaction (qPCR) has become the clinical gold standard for amplifying and detecting HPV DNA, existing molecular assays exhibit significant diagnostic and technical shortcomings. The extreme genetic diversity among the over 200 recognized HPV genotypes makes universal primer design exceedingly challenging, frequently resulting in primer-template mismatches and false-negative reporting. More critically, the vast majority of conventional molecular assays rely exclusively on targeting the highly conserved L1 structural gene. During the biological progression of HPV-induced oncogenesis, the circular viral episome frequently integrates into the host cell genome. This integration event regularly results in the targeted deletion or structural disruption of the L1 gene locus. Consequently, diagnostic assays relying solely on L1 amplification are inherently prone to producing false-negative results in patients with advanced, high-grade cervical intraepithelial neoplasia (CIN) or invasive carcinomas—precisely the clinical stage when accurate detection is most critical.

Furthermore, existing qPCR methodologies struggle with the complex dynamics of highly multiplexed genotyping. Clinical specimens frequently harbor concurrent co-infections with multiple, distinct HPV genotypes. In standard, highly pooled multiplex reactions, target competition for finite assay reagents (such as Taq polymerase and dNTPs) often leads to the preferential amplification of the most abundant viral strain, potentially masking the presence of clinically significant, lower-titer co-infecting strains. Additionally, owing to the inherent optical channel limitations of standard thermal cyclers (typically restricted to 4 to 6 fluorophores), current commercial assays are generally limited to uniquely differentiating only a small subset of high-risk genotypes (e.g., HPV 16 and 18), pooling the remainder into a single, non-differentiated positive result. This lack of comprehensive, high-resolution genotyping limits the physician's ability to track persistent, type-specific infections over time, leading to the potential overdiagnosis or clinical mismanagement of transient infections.

Finally, the inherent presence of potent PCR inhibitors in complex biological samples (such as blood, cervical mucus, or liquid-based cytology transport media) frequently causes assay amplification failure. In the absence of a rigorously integrated, endogenous internal control within each reaction, such localized failures result in undetected, false-negative clinical reports.

Thus, there remains a substantial and unmet clinical need for improved, highly multiplexed molecular diagnostic architectures capable of providing comprehensive, simultaneous genotyping of a vast plurality of HPV strains. Specifically, there is a need for robust diagnostic methods that strategically target multiple, complementary viral gene loci—such as synergistic combinations of structural and early oncogenic genes—to completely circumvent the false negatives associated with viral integration, while incorporating robust internal controls to guarantee assay validity on a reaction-by-reaction basis.

### SUMMARY OF THE INVENTION

According to a first aspect of the present application, there is provided an in vitro method for the highly multiplexed molecular genotyping and simultaneous detection of Human Papillomaviruses (HPVs) in a biological sample via real-time polymerase chain reaction (qPCR). The method comprises the physical steps of: (a) providing a biological sample containing, or suspected of containing, target viral nucleic acids; (b) partitioning said sample into a coordinated reaction set comprising eight physically discrete reaction pools (e.g., an 8-well strip or multi-well plate format). Each of the eight pools comprises a distinctly formulated multiplex qPCR master mix containing a specific subset of viral gene loci-specific oligonucleotides and sequence-specific fluorescent DNA probes. These oligonucleotides are strategically configured to target synergistic combinations of viral gene regions—preferably encompassing the highly conserved L1 structural gene along with the highly expressed E4 and integratable E6 early oncogenes; (c) subjecting the eight reaction pools simultaneously to a defined qPCR thermal cycling profile under stringent amplification conditions to generate target-specific amplicons; and (d) continuously monitoring and detecting the fluorescent optical signals emitted across multiple distinct detection channels within each of the eight pools, thereby enabling the simultaneous identification, differentiation, and clinical genotyping of up to 43 distinct HPV subtypes from a single sample.

In preferred embodiments of this method, the strategic distribution and grouping of the specific HPV targets across the eight individual pools is structurally optimized to reflect clinical prevalence and established co-infection statistics. Specifically, the oligonucleotides targeting the most clinically prevalent, frequently co-infecting high-risk HPV genotypes are deliberately segregated into separate reaction pools or assigned to non-overlapping optical channels. This optimized pooling architecture actively mitigates target competition for amplification reagents and substantially eliminates optical signal cross-talk, thereby ensuring maximum analytical sensitivity and ensuring the reliable detection of lower-titer strains in complex, mixed-genotype infections.

In a preferred embodiment, the highly multiplexed methods, reaction sets, and diagnostic kits of the present application are specifically configured for the simultaneous amplification, detection, differentiation, and clinical genotyping of up to 43 distinct Human Papillomavirus (HPV) genotypes within a single biological sample.

To provide comprehensive oncogenic risk stratification and directly guide patient clinical management, the 43 targeted HPV genotypes are systematically classified into three distinct clinical risk categories based on their epidemiological association with cervical dysplasias and carcinomas. In certain exemplary embodiments, the assay is configured to identify and differentiate targets selected from the following three categories:
(i) High-Risk Types (HRT): Comprising established, highly oncogenic genotypes unequivocally linked to invasive cervical cancer. This category includes 12 specific genotypes: HPV 16, 18, 31, 33, 35, 39, 45, 51, 52, 56, 58, and 59.
(ii) Probable/Possible Carcinogenic Types (PCT): Comprising intermediate-risk or putative high-risk genotypes that exhibit a probable epidemiological association with oncogenesis. This category includes 12 specific genotypes: HPV 26, 30, 34, 53, 66, 67, 68, 69, 70, 73, 82, and 85.
(iii) Low-Risk Types (LRT): Comprising genotypes primarily associated with benign epithelial lesions, such as anogenital warts (Condylomata acuminata), and rarely associated with invasive carcinomas. This category includes 19 specific genotypes: HPV 6, 11, 40, 42, 43, 44, 54, 55, 57, 61, 62, 64, 71, 72, 81, 83, 84, 89, and 90.

By explicitly differentiating between the HRT, PCT, and LRT categories on a reaction-by-reaction basis, the disclosed multiplex assay prevents the clinical overdiagnosis of transient, low-risk infections while ensuring the rigorous identification of high-risk and probable-risk viral strains, thereby maximizing the diagnostic and prognostic value of the test.

In a preferred embodiment, the diagnostic method and associated coordinated reaction set are configured to achieve the comprehensive detection and precise clinical genotyping of a 43-genotype HPV panel from a single biological sample.

To optimize both analytical multiplexing performance and downstream clinical reporting, the partitioning of targets in step (b) comprises the strategic segregation and combinatorial pooling of the HPV targets based on their established clinical risk categories. Specifically, the viral targets are grouped and allocated into the multiplex qPCR reaction pools according to their classification as High-Risk Types (HRT), Probable/Possible Carcinogenic Types (PCT), or Low-Risk Types (LRT). By intelligently allocating clinically related HPV targets into designated multiplex pools, the assay architecture streamlines diagnostic readouts, allowing clinicians to immediately assess a patient's oncogenic risk profile based upon which specific pool and corresponding fluorescent channel generates a positive amplification signal.

In this exemplary 43-genotype embodiment, the coordinated reaction set comprises exactly eight physically discrete multiplex qPCR reaction pools. Crucially, the total panel of 43 HPV genotypes is systematically distributed across these eight pools. Each individual reaction pool comprises a specifically formulated sub-mixture of the viral gene loci-specific oligonucleotides and sequence-specific fluorescent DNA probes (as provided in step a) designed to amplify and detect only a designated subset of the 43 genotypes, alongside the endogenous internal control (e.g., HBB). This distributed 8-pool architecture, combined with multi-channel fluorescent detection within each discrete pool, successfully overcomes the optical channel limitations of standard thermal cyclers while rigorously preventing target competition and reagent exhaustion among the 43 distinct viral strains.

In certain exemplary embodiments, the sequence-specific fluorescent DNA probes utilized in the multiplex qPCR reactions are dual-labeled hydrolysis probes. Each dual-labeled probe is covalently modified with a fluorescent reporter moiety (fluorophore) and a corresponding quenching moiety (quencher). These moieties are strictly selected to permit highly multiplexed optical detection with minimal to no spectral overlap. Preferably, the specific fluorophore-quencher pairs are selected from the group consisting of FAM-BHQ1, HEX-BHQ1, VIC-BHQ1, TAMRA-BHQ2, ROX-BHQ2, Texas Red-BHQ2, Cy5.5-BHQ2, Quasar 670-BHQ2, Cy5-BHQ2, and functionally equivalent combinations thereof.

The strategic selection and combinatorial assignment of these dual-labeled fluorescent DNA probes are critical to the multiplex architecture of the assay. Within each discrete multiplex reaction pool, distinct fluorophores possessing sufficiently separated excitation and emission spectra are conjugated to specific target probes. This precise optical arrangement ensures that the amplification of each targeted HPV genotype—or a defined clinical risk category of genotypes—generates a distinct, uniquely identifiable fluorescent signal. Consequently, the disclosed system enables the simultaneous detection, optical isolation, and precise clinical differentiation of multiple HPV genotypes within the same biological sample while actively mitigating optical cross-talk or signal interference between the designated detection channels.

In a preferred, highly advantageous embodiment, the viral gene loci-specific oligonucleotides (e.g., forward and reverse primers) and their corresponding fluorescent DNA probes are designed to be substantially complementary to, and specifically hybridize with, a synergistic combination of viral structural protein genes and viral regulatory/oncogenic protein genes. Specifically, the amplification targets preferably comprise the highly conserved L1 structural gene in concurrent combination with the E6 viral oncogene, alongside the strategic incorporation of the E4 gene target depending on its specific clinical significance for the designated HPV genotype. This multi-genic targeting strategy is specifically engineered to overcome the biological and clinical limitations of conventional single-target assays. The L1 major capsid protein gene provides a highly conserved baseline target that enables broad, pan-genotype detection. Crucially, the concurrent targeting of the E6 viral oncogene-which reliably remains integrated and actively transcribed within the host cell genome even when the L1 region is deleted during malignant progression-acts as an essential diagnostic fail-safe against false-negative reports in patients presenting with advanced precancerous lesions or invasive carcinomas. Furthermore, depending on the clinical significance and expression profile of the specific viral strain being targeted, the selective incorporation of the E4 gene provides an analytically robust secondary amplification locus. Because the E4 protein is heavily expressed during the productive phase of the viral life cycle in the epithelium, targeting the E4 locus maximizes the overall analytical sensitivity and diagnostic redundancy of the highly multiplexed assay.

In preferred embodiments, the method further comprises the concurrent amplification and detection of an endogenous assay internal control, specifically targeting an endogenous human housekeeping gene, for example, the human beta-globin (HBB) gene. The detection of this HBB target within at least one of the multiplex reaction pool serves to definitively verify sample adequacy, validate the nucleic acid extraction efficiency, and confirm the absence of localized PCR inhibitors, thereby ensuring the clinical validity of the diagnostic result on a reaction-by-reaction basis.

To achieve high-efficiency multiplexing, the qPCR reactions are conducted utilizing an optimized, high-fidelity master mix composition. This master mix comprises one or more of the following functional components: a thermostable DNA polymerase (preferably a hot-start DNA polymerase); a hot-start mechanism to prevent premature amplification and primer-dimer formation (such as polymerase-specific antibodies, aptamers, or chemical modifying agents); an optimized PCR reaction buffer (providing necessary divalent cations such as Mg²⁺); a comprehensive deoxynucleotide triphosphate (dNTP) mix comprising dATP, dCTP, dGTP, and dTTP (and/or dUTP utilized in combination with Uracil-DNA Glycosylase [UDG] for enzymatic carryover contamination prevention); nuclease-free or PCR-grade water; and optionally, a passive reference dye (such as ROX dye) to enable the normalization of non-PCR-related well-to-well fluorescent fluctuations. By way of example, but not limitation, such components may be sourced from commercial formulations such as the Qiagen QuantiNova® Probe PCR Kit or the Vazyme BioSmart™ U+ All-Powerful Multiple Probe qPCR PreMix. According to a second aspect of the present application, there is provided a diagnostic kit for the highly multiplexed molecular genotyping and simultaneous detection of HPVs in a biological sample in accordance with the disclosed methods. The kit physically comprises: (a) a plurality of viral gene loci-specific oligonucleotides and sequence-specific dual-labeled fluorescent DNA probes designed to target the L1, E4, and E6 regions of up to 43 distinct HPV genotypes; (b) internal control oligonucleotides targeting the HBB gene; and (c) the optimized qPCR master mix reagents. In preferred embodiments, the kit is pre-configured such that the oligonucleotides are spatially segregated into a coordinated reaction set (e.g., an 8-tube strip or multi-well plate consumable) to facilitate immediate clinical use.

According to another aspect of the present application, there is provided a kit for detecting and simultaneously genotyping Human Papillomaviruses (HPVs) in a biological sample, the kit comprises a plurality of physically discrete reaction pools configured to collectively detect and differentiate said plurality of HPV genotypes; wherein each reaction pool comprises a formulated, non-overlapping subset of viral gene loci-specific oligonucleotide triplets, each triplet comprising a forward primer, a reverse primer, and a sequence-specific dual-labeled fluorescent DNA probe; wherein the oligonucleotide triplets within the kit are configured to specifically hybridize to and amplify a synergistic combination of viral genetic targets, said targets comprising a highly conserved L1 structural gene and at least one early viral gene selected from the group consisting of an E4 gene and an E6 gene, thereby mitigating false-negative detection associated with viral episomal integration; and wherein at least one of said reaction pools further comprises an internal control primer-probe set targeting an endogenous human housekeeping gene to verify sample adequacy and the absence of amplification inhibitors. In some embodiments, the kit comprises exactly 8 physically discrete reaction pools, each reaction pool is configured to detect and genotype one of the following groups of HPV genotypes:
a first group of HPV genotypes: HPV genotypes 30, 33, 52, 62, 70, and 72;
a second group of HPV genotypes: HPV genotypes 11, 18, 51, 57, 64, 69, and 85;
a third group of HPV genotypes: HPV genotypes 31, 34, 42, 53, and 90;
a fourth group of HPV genotypes: HPV genotypes 40, 44, 45, 54, 55, and 89;
a fifth group of HPV genotypes: HPV genotypes 26, 56, 58, 71, and 84;
a sixth group of HPV genotypes: HPV genotypes 59, 61, 68, 73, and 83;
a seventh group of HPV genotypes: HPV genotypes 6, 39, 43, 66, and 82; and
an eighth group of HPV genotypes: HPV genotypes 16, 35, 67, and 81.

According to yet another aspect of the present application, there is provided a method for detecting and simultaneously genotyping Human Papillomaviruses (HPVs) in a biological sample within a single reaction or a coordinated reaction set. The method comprises steps of: (a) partitioning the biological sample into a coordinated reaction set comprising a plurality of physically discrete reaction pools from the kit provided herein and contacting the biological sample with the subset of viral gene loci-specific oligonucleotide triplets comprised in the reaction pools to form reaction mixtures, each triplet comprising a forward primer, a reverse primer, and a sequence-specific dual-labeled fluorescent DNA probe; (b) subjecting the reaction mixtures in the plurality of physically discrete reaction pools simultaneously to a nucleic acid amplification reaction under a PCR thermal cycling profile to generate target-specific amplicons; and (c) simultaneously detecting fluorescent optical signals emitted across multiple distinct detection channels within each of the reaction pools during the amplification reaction, thereby identifying and differentiating the HPV genotypes present in the biological sample. Each reaction pool is configured to detect and genotype one of the first to the eighth group of HPV genotypes as mentioned above.

According to another aspect of the present application, there is provided an automated diagnostic system for molecular genotyping of HPVs, the system comprises the kit described herein, a multi-channel real-time thermal cycling apparatus configured to execute the defined thermal cycling profile, and a computer-readable medium programmed with algorithmic logic to interpret the generated fluorescent signals from the eight discrete reaction pools and automatically classify the detected HPV targets into predefined clinical risk categories.

According to a yet aspect of the present application, there is provided an integrated real-time PCR diagnostic system configured to execute the molecular genotyping methods described herein. The system comprises: (a) the biochemical diagnostic kit components aforementioned; (b) a thermal cycling apparatus capable of executing stringent amplification profiles across a coordinated reaction set; (c) a multi-channel optical detection module configured to simultaneously excite and precisely read the emission spectra of multiple distinct fluorescent reporter moieties without significant spectral cross-talk; and (d) a computer-readable medium or software processor programmed with an algorithmic logic to interpret the generated fluorescent signals and automatically classify the detected HPV targets into defined clinical risk categories (e.g., High-Risk, Probable-Risk, and Low-Risk types).

### BRIEF DESCRIPTION OF THE DRAWINGS

The present application will now be described, by way of reference only, with respect to the accompanying figures:

FIG. 1A is the first part of a table summarizing the oligonucleotides and fluorescent dual-labeled probes used in accordance with an embodiment of the application;

FIG. 1B is the second part of a table summarizing the oligonucleotides and fluorescent dual-labeled probes used in accordance with an embodiment of the application;

FIG. 2 is a table summarizing the HPV genotypes in pool 1 to 8, risk types (carcinogenicity) and their fluorescent/quencher signals;

FIG. 3 is a schematic illustration showing qPCR reaction pools from 1 to 8 in an 8-tube strip format, each with final volume of 15 μL comprising 6 μL of 2× master mix, 6 μL of oligonucleotides and probes, 3 μL of sample extracted nucleic acid templates; the tube size or plate size should be limited 0.1 mL format;

FIG. 4A shows the cycling profiles used in the Bio-Rad Laboratories instruments in accordance with an embodiment of the application;

FIG. 4B shows the cycling profiles used in the Thermo Fisher Scientific instruments in accordance with an embodiment of the application;

FIG. 5A is a table summarizing the concentration of oligonucleotides and probes in pools 1 to 4;

FIG. 5B is a table summarizing the concentration of oligonucleotides and probes in pools 5 to 8;

FIG. 6A is a table summarizing the Hybribio immunoblot result, the corresponding blot image, the 43-genotype HPV panel Ct values for all detected genotypes, and commentary on assay-specific discrepancies for cases in accordance with an embodiment of the application;

FIG. 6B is a continued part of the table shown in FIG. 6A;

FIG. 7 is a table summarizing the genotypes detectable by the Hybribio 37-type assay versus those detectable by the 43-genotype HPV panel; and

FIG. 8 is a diagram illustrating the location of specific HPV probes on the HybriMem membrane of the Hybribio 37 HPV GenoArray Diagnostic Kit.

### DESCRIPTION OF THE PREFERRED EMBODIMENTS

Unless otherwise specifically provided, all ambient sample preparation and general handling herein are conducted at standard laboratory conditions which include a room temperature of approximately 25° C., sea level (1 atm.) pressure, standard non-condensing relative humidity, and pH 7. It is understood that actual amplification, hybridization, and thermal cycling steps are conducted at their respective protocol temperatures. Furthermore, all percentages, ratios, and concentrations herein are by weight-per-volume (w/v) or volume-per-volume (v/v) for liquid reagents, and molarity for oligonucleotides, unless specifically indicated otherwise. All aqueous solutions utilize standard molecular-grade, nuclease-free water. It is understood that unless otherwise specifically noted, the materials, compounds, chemicals, etc. described herein are typically commodity items and/or industry-standard items available from a variety of suppliers worldwide.

This application presents a multiplex real-time PCR (polymerase chain reaction) assay composition for the simultaneous detection of 43 different genotypes of human papillomaviruses (HPVs) in test samples. In some embodiments, the application covers the detection of three categories of HPV viruses that according to their carcinogenicity and pathogenicity, they are high risk types (HRT: 16, 18, 31, 33, 35, 39, 45, 51, 52, 56, 58 and 59), low risk types (LRT: 6, 11, 40, 42, 43, 44, 54, 55, 57, 61, 62, 64, 71, 72, 81, 83, 84, 89 and 90) and probable/possible carcinogenic types (PCT: 26, 30, 34, 53, 66, 67, 68, 69, 70, 73, 82 and 85). The composition comprises a variety of combinations of oligonucleotide sequences of forward/reverse primers and fluorescent dual ends labeled probes.

The multiplexing approach as disclosed herein involves the precision design of oligonucleotides that target the specific sites from the HPV viral genomes and from the human endogenous gene for assay internal control. This disclosure also addresses the selection of specific combination of HPV genotypes that are being detected or co-amplified in the same reactions and their corresponding signal of fluorescent colours. This disclosure also describes the specific amplification conditions and PCR master mix recipes that are employed to carry out the detection and genotyping of HPV viruses.

This application also presents an innovative method for the simultaneous detection of multiple Human Papillomavirus (HPV) types using real-time PCR. It is believed that the described approach enhances diagnostic accuracy and efficiency compared to conventional methods, enabling the detection of multiple HPV strains in several reactions with higher sensitivity and specificity. It is believed that this development addresses the limitations of traditional techniques by providing a more cost-effective and time-efficient solution for HPV genotyping and/or detection.

Without wishing to be bound by theory, this application highlights the applicability and practicability of HPV detection and genotyping by using real-time PCR technology for the testing of clinical specimens. It is believed that the advantages of real-time, fluorescent-labeled probe-based PCR enables the shortening of turnaround time for clinical diagnosis and offer exceptional performance when compared to traditional reverse dot-blot typing techniques. In addition, by considering the latest PCR multiplexing technology, the laboratory testing cost would then be greatly lowered and without scarifying the number of targets (HPV genotypes) that are being tested. It is believed that the HPV real-time PCR assay as described herein also simplifies the workflow and pipeline that is required in traditional testing methodology, reducing the training effort and manpower input in routine molecular laboratory settings.

### 1. Definitions

Unless otherwise specified, technical terms are used according to conventional usage in the art of molecular biology and molecular diagnostics.

As used herein, the term “primer” refers to an isolated oligonucleotide, whether occurring naturally or produced synthetically, that is capable of acting as a point of initiation of nucleic acid synthesis when placed under conditions in which synthesis of a primer extension product complementary to a target nucleic acid strand is induced. Such conditions typically include the presence of four different nucleoside triphosphates and an appropriate polymerase (e.g., a thermostable DNA polymerase) in an appropriate buffer and at a suitable temperature. A primer is preferably single-stranded for maximum efficiency in amplification.

As used herein, the terms “forward primer” and “reverse primer” refer to an oligonucleotide primer pair designed to amplify a specific target sequence. The “forward primer” is designed to hybridize to the anti-sense strand of the target double-stranded nucleic acid, while the “reverse primer” is designed to hybridize to the sense strand of the target. Together, under amplification conditions, they define the boundaries of the resulting amplicon.

As used herein, the term “viral gene loci-specific oligonucleotides” refers to a naturally occurring or synthetically produced sequence of nucleic acids (such as, but not limited to, amplification primers or detection probes) that is designed to, or is capable of, hybridizing to a specific, defined genetic region (locus) of a target viral genome under stringent amplification or hybridization conditions. Such oligonucleotides are substantially complementary to the designated target viral locus, allowing for the specific amplification, detection, or quantification of that particular viral sequence while exhibiting minimal or no cross-reactivity with host genomic nucleic acids, non-targeted commensal flora, or non-targeted viral genotypes. In the context of the present application, these oligonucleotides are specifically configured to target distinct loci of the human papillomavirus (HPV) genome—such as the L1, E4, or E6 regions—thereby enabling the precise identification and differentiation of specific viral strains in a sample.

As used herein, the term “probe” refers to an isolated oligonucleotide containing a detectable label or reporter molecule. A probe is designed to hybridize specifically to a target sequence within the amplicon generated by the forward and reverse primers. In the context of real-time PCR, the probe may be a hydrolysis probe (e.g., a TaqMan® probe) comprising a fluorophore at one terminus and a quenching moiety at the other.

As used herein, the terms “fluorescent moiety”, “fluorescent reporter” and “fluorescent reporter moiety” are used interchangeably, and refer to a chemical compound, dye, or functional group attached to an oligonucleotide (such as a hydrolysis probe or primer) that absorbs light energy at a specific excitation wavelength and emits light energy at a distinct, longer emission wavelength. The emitted light is detectable by an optical measurement system and serves as a quantifiable indicator of target nucleic acid amplification. Examples include, but are not limited to, fluorescein and its derivatives (e.g., FAM, HEX, TET, VIC), cyanine dyes (e.g., Cy3, Cy5), rhodamine derivatives (e.g., ROX, Texas Red), and other proprietary fluorescent dyes. In multiplex real-time PCR assays designed to detect and uniquely differentiate a large number of distinct viral genotypes within the same reaction or diagnostic kit, multiple probes are labeled with distinct fluorescent moieties possessing sufficiently separated emission spectra. This separation ensures simultaneous detection of multiple targets while minimizing optical overlap or signal interference (cross-talk).

As used herein, the term “quencher moiety” or “quenching molecule” refers to a molecule, chemical group, or compound attached to an oligonucleotide (typically a detection probe) that is capable of absorbing, dissipating, or otherwise substantially reducing the fluorescence emission of a spatially proximal fluorescent reporter moiety. This quenching effect typically occurs through mechanisms such as Fluorescence Resonance Energy Transfer (FRET), contact quenching, or static quenching. In the context of the present application, such as in hydrolysis probes or molecular beacons used in real-time PCR, the quencher moiety prevents the detection of a fluorescent signal while the oligonucleotide is intact. Upon hybridization to the target viral nucleic acid and subsequent cleavage by the 5′ to 3′ exonuclease activity of a polymerase during the amplification cycle, the fluorescent moiety is spatially separated from the quencher moiety. This separation abolishes the quenching effect, thereby generating a detectable and quantifiable fluorescent signal that is proportional to the amount of target amplicon produced. Examples of quencher moieties include, but are not limited to, Black Hole Quenchers™ (e.g., BHQ-1, BHQ-2, BHQ-3), Iowa Black® (FQ, RQ), DABCYL, TAMRA, QSY dyes, and Eclipse® quenchers.

As used herein, the term “fluorescent DNA probe” refers to an isolated oligonucleotide sequence, typically composed of deoxyribonucleic acid (DNA), that is covalently or non-covalently linked to at least one fluorescent reporter moiety. The oligonucleotide sequence is designed to be substantially complementary to, and hybridize with, a specific target nucleic acid sequence within an amplicon under stringent hybridization conditions. Upon binding to the target sequence, or upon undergoing a subsequent enzymatic cleavage or conformational change, the fluorescent DNA probe facilitates the emission of a detectable optical signal. This signal corresponds to the presence, absence, and/or quantity of the specific target sequence, such as a specific viral genotype, within a biological sample.

As used herein, the term “dual-labeled fluorescent DNA probe” refers to a specific embodiment of a fluorescent DNA probe that is covalently modified with two functionally distinct moieties: a fluorescent reporter moiety (fluorophore) and a quenching moiety (quencher). Typically, the fluorophore is attached to the 5′ terminus of the oligonucleotide and the quencher is attached to the 3′ terminus, although internal labeling of either moiety is also expressly encompassed herein. The dual-labeled fluorescent DNA probe relies on the spatial proximity of the fluorophore and quencher to suppress fluorescence emission while the oligonucleotide remains intact. During real-time amplification, the hybridization of the probe to its specific target nucleic acid, followed by enzymatic degradation (e.g., via the 5′ to 3′ exonuclease activity of a polymerase), spatially separates the fluorophore from the quencher. This separation yields a measurable and quantifiable increase in fluorescence. Such probes are highly advantageous in highly multiplexed molecular assays, such as those designed to simultaneously detect and differentiate up to 43 distinct viral subtypes across parallel reaction systems.

As used herein, the term “real-time PCR” or “qPCR” (real-time quantitative polymerase chain reaction) refers to a molecular biology methodology for the continuous monitoring, simultaneous amplification, and detection of a targeted nucleic acid molecule. Unlike traditional end-point PCR, which detects the accumulated amplicon only after the reaction is complete, qPCR utilizes fluorescent reporter molecules—such as dual-labeled fluorescent DNA probes—to generate a measurable optical signal that increases in direct proportion to the amount of PCR product generated during each successive thermal cycle. The cycle at which the fluorescent signal reliably exceeds a defined background fluorescence threshold is referred to as the cycle threshold (Ct) or quantification cycle (Cq), allowing for both the qualitative detection and absolute or relative quantification of the initial target nucleic acid concentration in a biological sample.

As used herein, the term “multiplex qPCR reactions” refers to a highly parallelized real-time qPCR assay configured to simultaneously amplify, detect, and uniquely differentiate multiple distinct target nucleic acid sequences within the same physical reaction mixture (e.g., within a single tube, well, or reaction chamber). This is accomplished by utilizing multiple, specifically designed primer pairs and corresponding sequence-specific detection probes, wherein the probes corresponding to different target loci are conjugated to distinct fluorescent reporter moieties with sufficiently separated, non-overlapping emission spectra. In the context of the present application, multiplex qPCR reactions enable the concurrent identification and diagnostic genotyping of numerous distinct viral strains—such as high-risk and low-risk HPV subtypes—alongside necessary internal quality controls in a single diagnostic run, thereby conserving biological sample volume, reducing reagent consumption, and increasing clinical throughput.

As used herein, the term “HPV target” refers to a specific, defined deoxyribonucleic acid (DNA) sequence or genomic region within the double-stranded Human Papillomavirus (HPV) genome that is intended to be amplified, detected, and/or quantified by the oligonucleotides, assays, and diagnostic kits of the present application. An HPV target may comprise a full-length viral gene, a partial gene sequence, or a specific genetic locus that is strictly conserved across multiple HPV strains or, conversely, is highly unique to a specific HPV genotype. Examples of preferred HPV DNA targets include, but are not limited to, the highly conserved L1 (major capsid protein) gene, as well as the E4 and E6 viral oncogene regions. In the context of the highly multiplexed methodologies described herein, an “HPV target” expressly encompasses the distinct genomic DNA sequence variations that enable the simultaneous identification, differentiation, and clinical genotyping of up to 43 distinct high-risk, intermediate-risk, and low-risk HPV subtypes from a single biological sample.

As used herein, the term “HPV Genotyping” refers to the specific identification, differentiation, and classification of distinct Human Papillomavirus (HPV) strains, subtypes, or genotypes present within a biological sample. Unlike broader HPV detection, genotyping involves utilizing uniquely designed, genotype-specific oligonucleotides (such as multiplexed primer pairs and differentially labeled fluorescent DNA probes) to explicitly distinguish between genetic sequence variations among different viral targets. In the context of the present application, HPV genotyping encompasses the simultaneous and specific identification of up to 43 distinct HPV DNA subtypes—including high-risk, intermediate-risk, and low-risk genotypes—within a highly multiplexed real-time PCR reaction system.

As used herein, the term “HPV detection” refers to the analytical process of determining the presence or absence of Human Papillomavirus (HPV) target deoxyribonucleic acid (DNA) within a biological sample. In the context of the present application, this involves the qualitative or quantitative measurement of an amplified target sequence to establish that at least one HPV viral strain is present in the sample, without necessarily specifying or differentiating the exact viral subtype.

As used herein, the term “sample” generally refers to any biological material being tested for and/or suspected of containing an analyte of interest, such as Human Papillomavirus (HPV) target deoxyribonucleic acid (DNA). Biological material includes all clinical specimens useful for the detection of HPVs in subjects, including, but not limited to, cells, tissues (e.g., obtained from a cervical swab, vaginal swab, endocervical brush, or tissue biopsy), and bodily fluids such as urine. Furthermore, the term expressly encompasses cells collected and suspended in liquid-based cytology (LBC) media (e.g., ThinPrep® PreservCyt® Solution, SurePath™ Preservative Fluid, or similar generic transport media), as well as specimens collected from other anatomical sites susceptible to HPV infection, such as the anogenital tract or oropharyngeal region. Importantly, for the purposes of the amplification and detection methodologies described herein, the term “sample” also explicitly includes any processed, fractionated, or purified derivatives of the aforementioned biological materials, such as crude cell lysates or isolated genomic viral DNA, that are subsequently introduced into the real-time PCR reaction mixture.

As used herein, the terms “hybridize” and “hybridization” refer to the physical and chemical process by which a single-stranded deoxyribonucleic acid (DNA) molecule joins with a substantially complementary single-stranded DNA molecule through sequence-specific base pairing (e.g., Watson-Crick hydrogen bonding) to form a stable, double-stranded nucleic acid duplex. In the context of the present application, “hybridization” specifically describes the highly specific binding of viral gene loci-specific oligonucleotides—such as forward/reverse amplification primers or dual-labeled fluorescent DNA probes—to their designated, complementary HPV target DNA sequences. This process occurs under defined, stringent amplification or hybridization conditions (including specific thermal cycling temperatures, buffer compositions, salt concentrations, and pH levels) that thermodynamically favor exact or highly homologous sequence matches. Consequently, successful hybridization under these stringent conditions ensures the precise targeted amplification and detection of specific viral genotypes while strictly minimizing or preventing non-specific binding (cross-reactivity) to non-target nucleic acids, such as host human genomic DNA, commensal microbial flora, or non-targeted HPV genotypes within the multiplex reaction.

As used herein, the term “human beta globin (HBB) gene” refers to the naturally occurring human genomic sequence encoding the beta-globin protein, located on human chromosome 11, or any defined deoxyribonucleic acid (DNA) target locus, fragment, or allele thereof. In the context of the present application, a specific target sequence within the HBB gene is utilized as an endogenous internal control (IC) or reference gene. The concurrent amplification and detection of this HBB target—utilizing specifically designed, HBB-directed oligonucleotides (such as forward/reverse primers and a distinctly labeled fluorescent DNA probe)—serves to validate the integrity of the diagnostic assay. Specifically, successful detection of the HBB target sequence verifies the presence of an adequate quantity of human cellular material in the biological sample (e.g., confirming proper swab collection), validates the efficacy of the DNA extraction and purification process, and confirms the absence of significant polymerase chain reaction (PCR) inhibitors that could otherwise result in a false-negative clinical report for HPV detection.

As used herein, a “single reaction” refers to an individual physical mixture (e.g., within a single microcentrifuge tube or a single well of a multi-well plate) capable of multiplexed detection utilizing distinct fluorescent channels. A “reaction set” refers to a coordinated plurality of individual, spatially separated reaction wells that collectively comprise the full diagnostic panel.

### 2. Primers and Probes

The present disclosure employs type-specific primers and probes configured to enable the simultaneous amplification and detection of the targeted HPV genotypes within a single reaction or a coordinated reaction set.

One aspect of this application relates to a specifically formulated, non-overlapping subset of viral gene loci-specific oligonucleotide triplets comprising a forward primer, a reverse primer, and a DNA probe. The forward and reverse primers, together with dual-labeled fluorescent probes, are designed to hybridize to selected regions of HPV genomic sequences. The HPV genome comprises viral structural protein genes (Late genes), specifically L1 and L2, as well as viral regulatory and oncogenic protein genes (Early genes), specifically E1, E2, E4, E5, E6, and E7. In exemplary embodiments, the primers and probes of the present application are configured to target one or more structural protein genes (L1 and L2), preferably the L1 gene, and/or one or more viral regulatory protein genes, preferably the E4 and E6 genes.

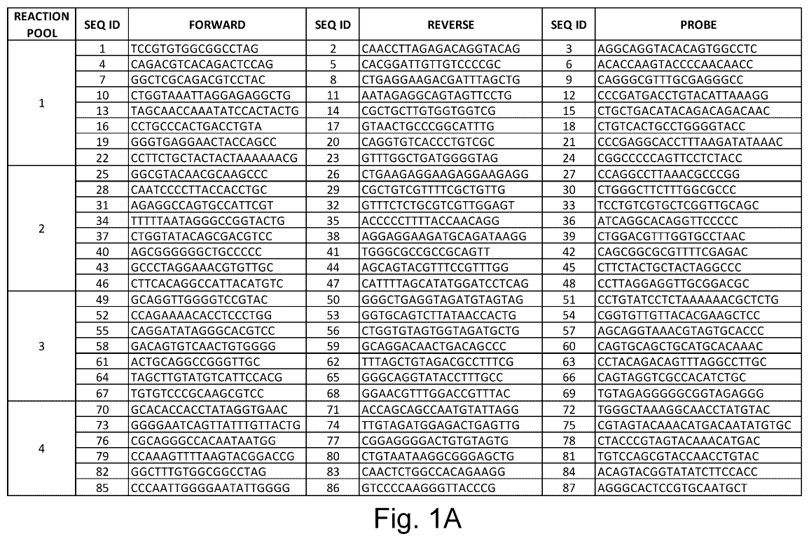

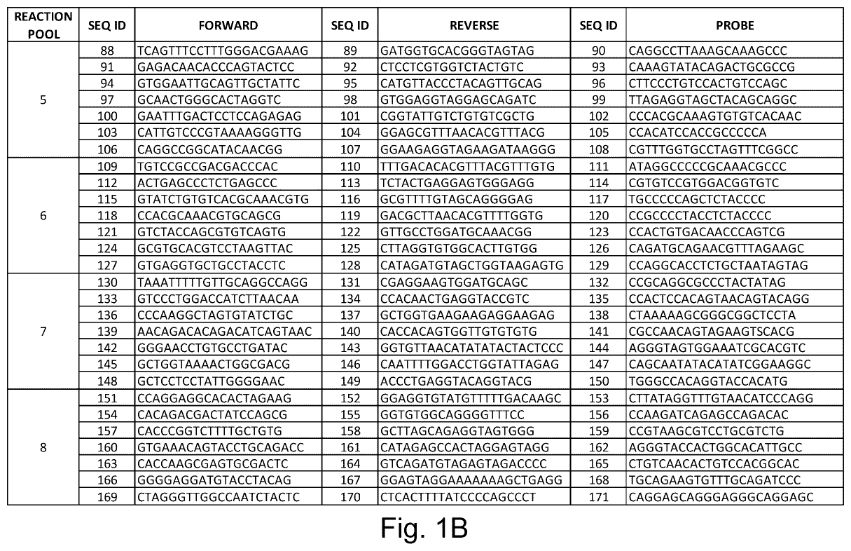

The present application provides highly optimized compositions, systems, and methods comprising strategically designed, viral gene loci-specific oligonucleotides and dual-labeled fluorescent DNA probes. Exemplary sequences, structural configurations, and target alignments of these oligonucleotides are illustrated in FIGS. 1A and 1B. The precise thermodynamic design of these primers and probes (including specific melting temperatures, GC content, and sequence length) enables highly stringent hybridization to their respective target sequences while preventing the formation of localized secondary structures or primer-dimers in a highly multiplexed environment.

In another embodiment, the triplets according to the present application comprises a forward primer (having a sequence comprising, consisting essentially of, or consisting of a nucleotide sequence SEQ ID No. 169, or a sequence having at least 90%, 95%, 98%, or 99% sequence identity thereto) and a reverse primer (having a sequence comprising, consisting essentially of, or consisting of a nucleotide sequence SEQ ID No. 170, or a sequence having at least 90%, 95%, 98%, or 99% sequence identity thereto) for amplifying a human beta-globin (HBB) gene, and a probe (having a sequence comprising, consisting essentially of, or consisting of a nucleotide sequence SEQ ID No 171, or a sequence having at least 90%, 95%, 98%, or 99% sequence identity thereto) for detecting an amplification product of the HBB gene.

In exemplary embodiments, the viral gene loci-specific oligonucleotide triplets comprises at least one forward primer comprising, consisting essentially of, or consisting of a nucleotide sequence selected from the group consisting of SEQ ID NOs: 1, 4, 7, 10, 13, 16, 19, 22, 25, 28, 31, 34, 37, 40, 43, 46, 49, 52, 55, 58, 61, 64, 67, 70, 73, 76, 79, 82, 85, 88, 91, 94, 97, 100, 103, 106, 109, 112, 115, 118, 121, 124, 127, 130, 133, 136, 139, 142, 145, 148, 151, 154, 157, 160, 163, and 166, or a sequence having at least 90%, 95%, 98%, or 99% sequence identity thereto. In some embodiments, the forward primers are grouped in such a way that each group can be utilized to amplify a specific set of HPV genotypes. In exemplary embodiments, the forward primers described herein are coordinated into 8 groups as shown in FIGS. 1A and 1B.

In exemplary embodiments, the viral gene loci-specific oligonucleotide triplets comprises at least one reverse primer comprising, consisting essentially of, or consisting of a nucleotide sequence selected from the group consisting of SEQ ID NOs: 2, 5, 8, 11, 14, 17, 20, 23, 26, 29, 32, 35, 38, 41, 44, 47, 50, 53, 56, 59, 62, 65, 68, 71, 74, 77, 80, 83, 86, 89, 92, 95, 98, 101, 104, 107, 110, 113, 116, 119, 122, 125, 128, 131, 134, 137, 140, 143, 146, 149, 152, 155, 158, 161, 164, and 167, or a sequence having at least 90%, 95%, 98%, or 99% sequence identity thereto. In some embodiments, the reverse primers are grouped in such a way that each group can be utilized to amplify a specific set of HPV genotypes. In exemplary embodiments, the reverse primers described herein are coordinated into 8 groups as shown in FIGS. 1A and 1B.

In exemplary embodiments, the viral gene loci-specific oligonucleotide triplets comprises at least one DNA probe comprising, consisting essentially of, or consisting of a nucleotide sequence selected from the group consisting of SEQ ID NOs: 3, 6, 9, 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, 162, 165, and 168, or a sequence having at least 90%, 95%, 98%, or 99% sequence identity thereto. In some embodiments, the probes are grouped in such way that each group can be utilized to detect a specific set of HPV genotypes. In exemplary embodiments, the probes described herein are coordinated into 8 groups as shown in FIGS. 1A and 1B.

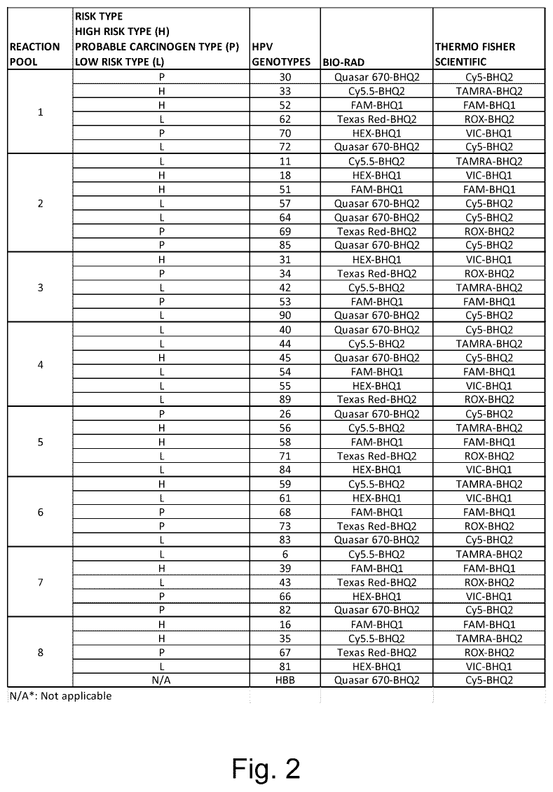

It is expressly understood that these oligonucleotides described above are configured to operate in functionally designated target-specific triplets (e.g., SEQ ID NOs: 1, 2, and 3 functioning concertedly to amplify and detect a first specific target locus). For example, the triplets shown in FIGS. 1A and 1B are grouped into eight reaction pools, each group is used for detecting and differentiating the HPV genotypes in the corresponding reaction pool as shown in FIG. 2. In other words, triplets in reaction pool 1 in FIGS. 1A and 1B are specific for HPV genotypes listed for reaction pool 1 in FIG. 2.

In some embodiments, the DNA probes herein comprises a fluorescent reporter moiety and a quencher moiety. The probes comprises fluorophore-quencher combinations selected from the group consisting of FAM-BHQ1, HEX-BHQ1, VIC-BHQ1, TAMRA-BHQ2, ROX-BHQ2, Texas Red-BHQ2, Cy5.5-BHQ2, Quasar 670-BHQ2, Cy5-BHQ2, and functionally equivalent fluorophore-quencher combinations thereof (including internal quenching arrangements), configured to generate distinct emission spectra without significant optical cross-talk. Each coordinated triplet is assigned to uniquely identify one or more specific HPV genotypes or genotype groups. Distributing these specific triplets across a coordinated reaction set enables the simultaneous detection of up to 43 distinct HPV targets while substantially avoiding signal interference or target competition between amplification channels. In exemplary embodiments, the probes are coordinated into eight groups, each of which is specific for one of the following groups of HPV genotypes:
a first group of HPV genotypes: HPV genotypes 30, 33, 52, 62, 70, and 72;
a second group of HPV genotypes: HPV genotypes 11, 18, 51, 57, 64, 69, and 85;
a third group of HPV genotypes: HPV genotypes 31, 34, 42, 53, and 90;
a fourth group of HPV genotypes: HPV genotypes 40, 44, 45, 54, 55, and 89;
a fifth group of HPV genotypes: HPV genotypes 26, 56, 58, 71, and 84;
a sixth group of HPV genotypes: HPV genotypes 59, 61, 68, 73, and 83;
a seventh group of HPV genotypes: HPV genotypes 6, 39, 43, 66, and 82; and
an eighth group of HPV genotypes: HPV genotypes 16, 35, 67, and 81.

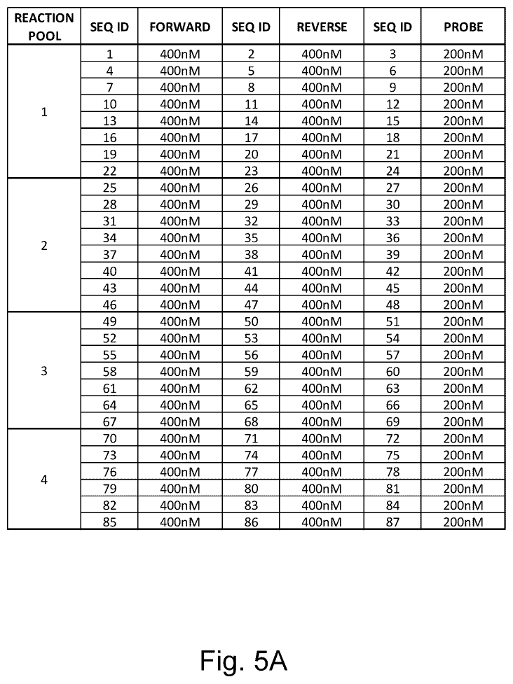

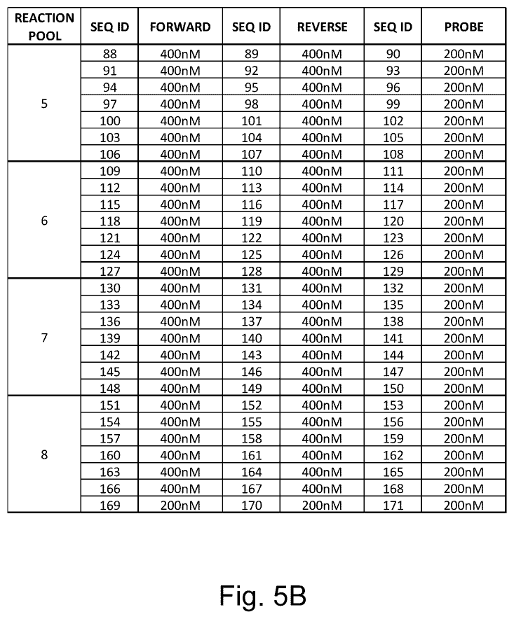

Representative primer and probe sequences, target pairings, and exemplary concentration profiles are provided in FIGS. 1A-1B and 5A-5B. Such examples are illustrative of preferred embodiments and are not intended to limit the scope of the disclosed multiplex architectures or compositions.

The primers and probes provided herein may be synthesized using any of standard methods conventionally known to those of ordinary skills in the art. For example, the primers and probes may be synthesized using standard solid-phase phosphoramidite triester chemistry. Unlabeled amplification primers (forward and reverse) are synthesized with standard deoxynucleotide phosphoramidites, cleaved from the solid support, deprotected, and purified to a high degree of homogeneity.

For the sequence-specific fluorescent probes, dual-labeling is achieved utilizing standard conjugation chemistries. The selected quenching moieties (e.g., Black Hole Quenchers such as BHQ-1, BHQ-2, or BHQ-3) are typically incorporated at the 3′ terminus during the initial stages of synthesis utilizing quencher-modified controlled pore glass (CPG) solid supports. The fluorescent reporter moieties (e.g., FAM, HEX, ROX, Cy5) are subsequently covalently conjugated to the 5′ terminus using commercially available fluorophore-modified phosphoramidites during the final automated synthesis cycle, or alternatively via post-synthetic N-hydroxysuccinimide (NHS) ester coupling chemistries.

Following synthesis and cleavage, all labeled probes and unlabeled primers are subjected to rigorous purification—preferably utilizing High-Performance Liquid Chromatography (HPLC) or Polyacrylamide Gel Electrophoresis (PAGE)—to remove truncated synthesis failure sequences and uncoupled free fluorophores. This strict purification ensures optimal analytical performance, maximum signal-to-noise ratios, and strict adherence to the manufacturing quality control standards required for clinical-grade diagnostic applications.

### 2. Kit

The present application provides comprehensive in vitro diagnostic kits specifically formulated and packaged for the highly multiplexed molecular genotyping, detection, and clinical risk stratification of up to 43 distinct Human Papillomavirus (HPV) genotypes within a biological sample.

One aspect of this application relates to a kit for detecting and simultaneously genotyping Human Papillomaviruses (HPVs) in a biological sample. The kit comprises a plurality of physically discrete reaction pools (for example, 2, 3, 4, 5, 6, 7 or 8 reaction pools) configured to collectively detect and differentiate said plurality of HPV genotypes, wherein each reaction pool comprises a specifically formulated, non-overlapping subset of viral gene loci-specific oligonucleotide triplets as described above, each triplet comprising a forward primer, a reverse primer, and a sequence-specific dual-labeled fluorescent DNA probe. The oligonucleotide triplets within the kit are configured to specifically hybridize to and amplify a synergistic combination of viral genetic targets, said targets comprising a highly conserved L1 structural gene and at least one early viral gene selected from the group consisting of an E4 gene and an E6 gene, thereby mitigating false-negative detection associated with viral episomal integration. At least one of the plurality of reaction pools further comprises an internal control primer-probe set targeting an endogenous human housekeeping gene to verify sample adequacy and the absence of amplification inhibitors.

In some embodiments, the plurality of physically discrete reaction pools consists of exactly 8 reaction pools, and wherein each reaction pool is configured to detect and genotype one of the following groups of HPV genotypes:
a first group of HPV genotypes: HPV genotypes 30, 33, 52, 62, 70, and 72;
a second group of HPV genotypes: HPV genotypes 11, 18, 51, 57, 64, 69, and 85;
a third group of HPV genotypes: HPV genotypes 31, 34, 42, 53, and 90;
a fourth group of HPV genotypes: HPV genotypes 40, 44, 45, 54, 55, and 89;
a fifth group of HPV genotypes: HPV genotypes 26, 56, 58, 71, and 84;
a sixth group of HPV genotypes: HPV genotypes 59, 61, 68, 73, and 83;
a seventh group of HPV genotypes: HPV genotypes 6, 39, 43, 66, and 82; and
an eighth group of HPV genotypes: HPV genotypes 16, 35, 67, and 81.

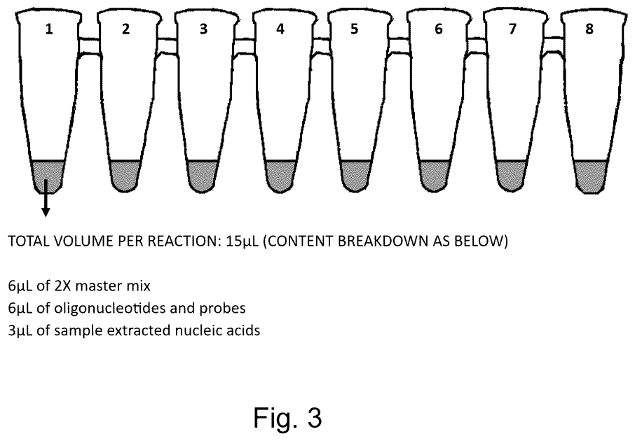

To overcome the optical limitations of standard real-time PCR instrumentation while achieving comprehensive clinical genotyping (e.g., a 43-genotype panel), the application comprises a deliberate multiplex combinatorial pooling strategy. As depicted in FIG. 3, specific subsets of the targeted viral genotypes are physically segregated and distributed into individual reaction wells across a coordinated reaction set. Without intending to be bound by theory, it is believed that this spatial pooling—combined with the simultaneous targeting of synergistic viral genes (e.g., combining the highly conserved L1 structural gene with the highly expressed E4 and integratable E6 early genes)—can maximize clinical sensitivity and mitigates target competition during amplification.

In some embodiments, the oligonucleotide triplets comprises a forward primer, a reverse primer and a DNA probe as described in the above section.

In some embodiments, each reaction pool of the kit according to this application comprises one or more forward primers (e.g., one, two, three, four, five, six or more forward primers) selected from one of the following groups:
a first group of forward primers, each comprising or consisting of a nucleotide sequence selected from the group consisting of SEQ ID NOs: 1, 4, 7, 10, 13, 16, 19, and 22, or a sequence having at least 90%, 95%, 98%, or 99% sequence identity thereto;
a second group of forward primers, each comprising, consisting essentially of, or consisting of a nucleotide sequence selected from the group consisting of SEQ ID NOs: 25, 28, 31, 34, 37, 40, 43, and 46, or a sequence having at least 90%, 95%, 98%, or 99% sequence identity thereto;
a third group of forward primers, each comprising or consisting of a nucleotide sequence selected from the group consisting of SEQ ID NOs: 49, 52, 55, 58, 61, 64, and 67, or a sequence having at least 90%, 95%, 98%, or 99% sequence identity thereto;
a fourth group of forward primers, each comprising, or consisting of a nucleotide sequence selected from the group consisting of SEQ ID NOs: 70, 73, 76, 79, 82, and 85, or a sequence having at least 90%, 95%, 98%, or 99% sequence identity thereto;
a fifth group of forward primers, each comprising or consisting of a nucleotide sequence selected from the group consisting of SEQ ID NOs: 88, 91, 94, 97, 100, 103, and 106, or a sequence having at least 90%, 95%, 98%, or 99% sequence identity thereto;
a sixth group of forward primers, each comprising or consisting of a nucleotide sequence selected from the group consisting of SEQ ID NOs: 109, 112, 115, 118, 121, 124, and 127, or a sequence having at least 90%, 95%, 98%, or 99% sequence identity thereto;
a seventh group of forward primers, each comprising or consisting of a nucleotide sequence selected from the group consisting of SEQ ID NOs: 130, 133, 136, 139, 142, 145, and 148, or a sequence having at least 90%, 95%, 98%, or 99% sequence identity thereto;
an eighth group of forward primers, each comprising or consisting of a nucleotide sequence selected from the group consisting of SEQ ID NOs: 151, 154, 157, 160, 163, 166, and 169, or a sequence having at least 90%, 95%, 98%, or 99% sequence identity thereto.

Alternatively, the kit comprises eight groups of forward primer, wherein each group contains the forward primers as described above. As an example, each triplet comprises a forward primer, a reverse primer and a DNA probe shown in each line of the table shown in FIGS. 1A and 1B.

In some embodiments, each reaction pool of the kit comprises one or more reverse primers selected from one of the following groups:
a first group of reverse primers, each comprising or consisting of a nucleotide sequence selected from the group consisting of SEQ ID NOs: 2, 5, 8, 11, 14, 17, 20, and 23, or a sequence having at least 90%, 95%, 98%, or 99% sequence identity thereto;
a second group of reverse primers, each comprising or consisting of a nucleotide sequence selected from the group consisting of SEQ ID NOs: 26, 29, 32, 35, 38, 41, 44, and 47, or a sequence having at least 90%, 95%, 98%, or 99% sequence identity thereto;
a third group of reverse primers, each comprising or consisting of a nucleotide sequence selected from the group consisting of SEQ ID NOs: 50, 53, 56, 59, 62, 65, and 68, or a sequence having at least 90%, 95%, 98%, or 99% sequence identity thereto;
a fourth group of reverse primers, each comprising, or consisting of a nucleotide sequence selected from the group consisting of SEQ ID NOs: 71, 74, 77, 80, 83, and 86, or a sequence having at least 90%, 95%, 98%, or 99% sequence identity thereto;
a fifth group of reverse primers, each comprising or consisting of a nucleotide sequence selected from the group consisting of SEQ ID NOs: 89, 92, 95, 98, 101, 104, and 107, or a sequence having at least 90%, 95%, 98%, or 99% sequence identity thereto;
a sixth group of reverse primers, each comprising or consisting of a nucleotide sequence selected from the group consisting of SEQ ID NOs: 110, 113, 116, 119, 122, 125, and 128, or a sequence having at least 90%, 95%, 98%, or 99% sequence identity thereto;
a seventh group of reverse primers, each comprising or consisting of a nucleotide sequence selected from the group consisting of SEQ ID NOs: 131, 134, 137, 140, 143, 146, and 149, or a sequence having at least 90%, 95%, 98%, or 99% sequence identity thereto; and
an eighth group of reverse primers, each comprising or consisting of a nucleotide sequence selected from the group consisting of SEQ ID NOs: 152, 155, 158, 161, 164, 167, and 170, or a sequence having at least 90%, 95%, 98%, or 99% sequence identity thereto.

Alternatively, the kit comprises eight groups of reverse primers, wherein each group contains the reverse primers as described above.

In some embodiments, each reaction pool of the kit comprises one or more probes selected from one of the following groups:
a first group of probes, each comprising or consisting of a nucleotide sequence selected from the group consisting of SEQ ID NOs: 3, 6, 9, 12, 15, 18, 21, 24, or a sequence having at least 90%, 95%, 98%, or 99% sequence identity thereto;
a second group of probes, each comprising or consisting of a nucleotide sequence selected from the group consisting of SEQ ID NOs: 27, 30, 33, 36, 39, 42, 45, 48, or a sequence having at least 90%, 95%, 98%, or 99% sequence identity thereto;
a third group of probes, each comprising or consisting of a nucleotide sequence selected from the group consisting of SEQ ID NOs: 51, 54, 57, 60, 63, 66, 69, or a sequence having at least 90%, 95%, 98%, or 99% sequence identity thereto;
a fourth group of probes, each comprising or consisting of a nucleotide sequence selected from the group consisting of SEQ ID NOs: 72, 75, 78, 81, 84, 87, or a sequence having at least 90%, 95%, 98%, or 99% sequence identity thereto;
a fifth group of probes, each comprising or consisting of a nucleotide sequence selected from the group consisting of SEQ ID NOs: 90, 93, 96, 99, 102, 105, 108, or a sequence having at least 90%, 95%, 98%, or 99% sequence identity thereto;
a sixth group of probes, each comprising or consisting of a nucleotide sequence selected from the group consisting of SEQ ID NOs: 111, 114, 117, 120, 123, 126, 129, or a sequence having at least 90%, 95%, 98%, or 99% sequence identity thereto;
a seventh group of probes, each comprising or consisting of a nucleotide sequence selected from the group consisting of SEQ ID NOs: 132, 135, 138, 141, 144, 147, 150, or a sequence having at least 90%, 95%, 98%, or 99% sequence identity thereto; and
an eighth group of probes, each comprising or consisting of a nucleotide sequence selected from the group consisting of SEQ ID NOs: 153, 156, 159, 162, 165, 168, and 171, or a sequence having at least 90%, 95%, 98%, or 99% sequence identity thereto.

Alternatively, the kit comprises eight groups of probes, wherein each group contains the probes as described above.

In some embodiments, the triplet is configured to target one structural protein gene, L1 gene, in combination with one or more viral regulatory genes selected from the group consisting of E4 and E6 genes of HPV genomic sequences.

In some embodiments, the kit comprise 8 reaction pools.

In preferred embodiments, the diagnostic kit physically comprises a systematically pooled mixture of viral gene loci-specific oligonucleotides. Crucially, these oligonucleotides are not provided as isolated components, but rather as functionally validated target-specific triplets. Each triplet comprises a designated forward primer, a corresponding reverse primer, and a sequence-specific dual-labeled fluorescent DNA probe, which are collectively configured to specifically hybridize with and amplify synergistic target regions (e.g., the L1, E4, and E6 genes) of a designated HPV genotype.

To facilitate immediate clinical laboratory implementation, the kit further comprises an optimized, high-fidelity qPCR master mix. This master mix provides the essential biochemical environment for highly multiplexed amplification and comprises: a thermostable DNA polymerase (preferably a hot-start polymerase); a comprehensive deoxynucleotide triphosphate (dNTP) mix (optionally including dUTP and Uracil-DNA Glycosylase [UDG] for enzymatic carryover contamination prevention); optimized buffer components providing essential divalent cations; and necessary reaction stabilizers. By way of non-limiting example, the kit may incorporate or be utilized in conjunction with commercial-grade formulations such as the QuantiNova® Probe PCR Kit (Qiagen) or the BioSmart™ U+ All-Powerful Multiple Probe qPCR PreMix (Vazyme).

To ensure robust multiplexing performance without exhausting reaction components, primer and probe concentrations in the master mix are strategically calibrated. Broadly, oligonucleotide concentrations may range from about 50 nM to about 1000 nM. More preferably, concentrations range from about 100 nM to about 600 nM. In exemplary, highly optimized embodiments, primer and probe concentrations are strictly calibrated to range from about 200 nM to about 400 nM to achieve maximum amplification efficiency and detection sensitivity while preventing non-specific fluorescence.

In exemplary embodiments, primer and probe concentrations are selected to achieve efficient amplification and detection, and may range, for example, from about 200 nM to about 400 nM, although other concentrations may be used depending on assay configuration. For example, the concentrations of primers and probes can be selected as shown in FIG. 5B.

In highly optimized commercial embodiments, the diagnostic kit is pre-configured into a coordinated reaction set comprising a plurality of physically discrete multiplex reaction pools (e.g., a pre-aliquoted 8-well strip or multi-well plate consumable). The total panel of targeted HPV genotypes is systematically divided and distributed across these discrete pools based on carcinogenic risk and prevalent co-infection patterns. Consequently, each discrete reaction well within the provided kit contains a uniquely formulated sub-mixture of the aforementioned oligonucleotide triplets, meticulously designed to detect a specific subset of the HPV panel without inducing localized target competition or optical signal interference.

Furthermore, to guarantee assay validity on a reaction-by-reaction basis—a critical requirement for stringent regulatory compliance—the kit is strictly provisioned with an endogenous internal control system. In some embodiments, the kit further comprises an internal control primer-probe set, which comprises an internal control primer for amplifying a human housekeeping gene (such as the human beta-globin [HBB] gene); and an internal control probe for detecting an amplification product of the housekeeping gene such as HBB gene. The control primer comprises an internal control forward primer comprising or consisting of a nucleotide sequence SEQ ID No. 169, or a sequence having at least 90%, 95%, 98%, or 99% sequence identity thereto; an internal control reverse primer comprising or consisting of a nucleotide sequence SEQ ID No. 170, or a sequence having at least 90%, 95%, 98%, or 99% sequence identity thereto. The internal control probe comprises or consists of a nucleotide sequence SEQ ID No. 171, or a sequence having at least 90%, 95%, 98%, or 99% sequence identity thereto. Oligonucleotide primers and probes configured to target a human housekeeping gene (such as the human beta-globin [HBB] gene) are uniformly incorporated into the master mix of at least one of the multiplex reaction pool provided within the kit, for example, every reaction pool, or only one of the reaction pools, e.g., the 8^{th} one of the total eight reaction pools). This ensures that the end-user can continuously verify sample cellular adequacy, nucleic acid extraction efficiency, and the absence of PCR inhibitors across all designated detection channels.

Another aspect of this application relates to a kit for molecular genotyping and detection of HPVs in a biological sample utilizing the method provided herein. The kit comprises: a plurality of sequence-specific oligonucleotide triplets targeting up to 43 distinct HPV genotypes; an endogenous internal control primer-probe set targeting the human beta-globin (HBB) gene; and optionally, an optimized qPCR master mix. In the kit, the components are pre-configured into a coordinated reaction consumable comprising exactly eight discrete multiplex reaction pools, each pool containing a distinct subset of the oligonucleotide triplets.

### 3. Method

One aspect of this application relates to a method for detecting and simultaneously genotyping Human Papillomaviruses (HPVs) in a biological sample within a single reaction or a coordinated reaction set. The method comprises the steps of:
(a) partitioning the biological sample into a coordinated reaction set comprising a plurality of physically discrete reaction pools (for example, 2, 3, 4, 5, 6, 7 or 8 reaction pools) and contacting the biological sample with the subset of viral gene loci-specific oligonucleotide triplets comprised in the reaction pools to form reaction mixtures, each triplet comprising a forward primer, a reverse primer, and a sequence-specific dual-labeled fluorescent DNA probe;
(b) subjecting the reaction mixtures in the plurality of physically discrete reaction pools simultaneously to a nucleic acid amplification reaction under a PCR thermal cycling profile to generate target-specific amplicons;
(c) simultaneously detecting fluorescent optical signals emitted across multiple distinct detection channels within each of the reaction pools during the amplification reaction, thereby identifying and differentiating the HPV genotypes present in the biological sample.

In some embodiments, the biological sample is partitioned into exactly 8 reaction pools, and each reaction pool is configured to detect and genotype one of the following groups of HPV genotypes:
a first group of HPV genotypes: HPV genotypes 30, 33, 52, 62, 70, and 72;
a second group of HPV genotypes: HPV genotypes 11, 18, 51, 57, 64, 69, and 85;
a third group of HPV genotypes: HPV genotypes 31, 34, 42, 53, and 90;
a fourth group of HPV genotypes: HPV genotypes 40, 44, 45, 54, 55, and 89;
a fifth group of HPV genotypes: HPV genotypes 26, 56, 58, 71, and 84;
a sixth group of HPV genotypes: HPV genotypes 59, 61, 68, 73, and 83;
a seventh group of HPV genotypes: HPV genotypes 6, 39, 43, 66, and 82; and
an eighth group of HPV genotypes: HPV genotypes 16, 35, 67, and 81.

The method can be performed using the kit provided herein.

In some embodiments, the biological sample is partitioned into eight reaction pools, wherein each reaction pool is configured to specifically detect and genotype for one of the first to the eighth group of HPV genotypes. Specifically, the eight reaction pools may comprise eight distinct groups of primers and eight distinct groups of probes that are specifically amplifying and detecting the eight groups of HPV genotypes.

In some embodiments, the probes hybridize to complementary sequences within the amplicons during step (b).

In specific embodiments, the method comprises a multiplex qPCR assay that employs a highly optimized plurality of viral type-specific primers and corresponding dual-labeled fluorescent probes. These oligonucleotides are strategically configured to enable the simultaneous amplification, optical detection, and precise clinical differentiation of the targeted HPV genotypes within a coordinated reaction set. As utilized herein, the “reaction set” comprises a predetermined number of spatially discrete multiplex reaction pools (e.g., an 8-well configuration), wherein the comprehensive panel of viral targets is systematically distributed to maximize the optical multiplexing capacity of the instrument while actively preventing reagent competition among the viral strains.

The application further encompasses the novel combinatorial architecture of the multiplex diagnostic assay. This includes, but is not limited to, the strategic assignment of distinct fluorescent reporter moieties and corresponding quenching molecules to establish separate, non-overlapping optical emission channels (e.g., as shown in FIG. 2). By assigning specific fluorescent channels to specific target categories, the assay uniquely differentiates each targeted HPV genotype.

In some embodiments, the structural distribution of the HPV targets across the exactly eight physically discrete multiplex qPCR reaction pools in step (a) is systematically organized based upon established clinical risk classifications and epidemiological co-infection prevalence to actively mitigate target competition for amplification reagents and prevent optical signal interference.

In some embodiments, the coordinated reaction set is configured to simultaneously detect and clinically differentiate up to 43 distinct HPV genotypes from the single biological sample. The 43 distinct HPV genotypes are strictly categorized into three clinical diagnostic risk tiers, comprising: High-Risk Types (16, 18, 31, 33, 35, 39, 45, 51, 52, 56, 58, and 59); Probable/Possible Carcinogenic Types (26, 30, 34, 53, 66, 67, 68, 69, 70, 73, 82, and 85); and Low-Risk Types (6, 11, 40, 42, 43, 44, 54, 55, 57, 61, 62, 64, 71, 72, 81, 83, 84, 89, and 90).

In some embodiments, the HPV genotypes are combined and allocated into the eight physically discrete multiplex qPCR reaction pools specifically according to their designated clinical diagnostic risk tier to streamline automated clinical risk reporting.

In certain embodiments, the viral gene loci-specific oligonucleotides and fluorescent DNA probes are engineered to specifically hybridize with and amplify a synergistic combination of viral genetic targets, specifically comprising the highly conserved structural L1 gene and the early oncogenic E6 gene, alongside the selective incorporation of the early E4 gene, thereby circumventing false-negative detection associated with viral episomal integration.

In some embodiments, the concurrent amplification and optical detection of an endogenous assay internal control targeting an endogenous human housekeeping gene (e.g., the human beta-globin (HBB) gene), wherein oligonucleotides targeting the human housekeeping gene (e.g., HBB gene) are incorporated in at least one of the plurality of reaction pools (for example, in every reaction pool, or in only one of the reaction pool, e.g., the 8^{th} one of the total eight reaction pools) to independently verify sample adequacy and the absence of PCR inhibitors on a reaction-by-reaction basis. In a preferred, highly advantageous embodiment, the multiplex qPCR assay further comprises a systematically integrated endogenous internal control system targeting a stably expressed human housekeeping gene, specifically the human beta-globin (HBB) gene. Crucially, the oligonucleotides targeting the HBB internal control are incorporated into the master mix of at least one of the reaction pools (e.g., the 8^{th} one of the total eight reaction pools) comprising the coordinated reaction set. The concurrent amplification and multi-channel detection of this HBB signal serves as an indispensable, assay-specific quality control metric. It definitively verifies the adequacy of the clinical sample collection (e.g., ensuring sufficient human cellularity from a swab), validates the extraction efficiency and integrity of the nucleic acids, and affirmatively confirms the absence of localized PCR inhibitors on a reaction-by-reaction basis.

To achieve the simultaneous identification and differentiation of up to 43 distinct HPV subtypes, the targets are systematically distributed across the reaction set. Each reaction pool within the set contains a highly multiplexed sub-mixture of type-specific primers and distinctly labeled fluorescent probes designed to identify a specific subset of the HPV genotypes, thereby maximizing the optical multiplexing capacity of standard real-time PCR instrumentation without optical cross-talk.

In preferred embodiments, the multiplex qPCR assay further comprises an endogenous internal control targeting a human housekeeping gene, such as the human beta-globin (HBB) gene, to continuously verify sample adequacy, nucleic acid integrity, and nucleic acid extraction efficiency. Crucially, the oligonucleotides directed to this internal control are incorporated into the master mix of each individual reaction well comprising the reaction set. The concurrent amplification and detection of the internal control signal within at least one of the reaction well confirms the continuous presence of amplifiable human DNA and serves as an independent, well-specific quality control. This systematic redundancy ensures that the absence of a viral target signal in any given well is a true negative result, affirmatively ruling out localized technical failures such as well-specific reagent dispensing errors, localized thermal cycling anomalies, or the presence of localized polymerase chain reaction (PCR) inhibitors.

In preferred embodiments, the multiplex diagnostic assay utilizes a plurality of target-specific amplification sets. Each set comprises a coordinated triplet: a forward primer, a reverse primer, and a sequence-specific fluorescent DNA probe. For example, as shown in FIGS. 1A and 1B, the forward and reverse primers and the probes are grouped into eight reaction pools. Moreover, each set is designed to amplify and detect a specific viral or endogenous control locus, for example, as shown in FIG. 2.

Finally, the application encompasses the rigorous optimization of the multiplex qPCR amplification parameters, including optimized master mix formulations, customized thermal cycling profiles, and calibrated primer-to-probe concentration ratios. These optimized parameters collectively ensure robust analytical performance, achieving high analytical sensitivity (Limit of Detection) and strict analytical specificity with minimal optical cross-talk, even in the presence of overwhelming background host genomic DNA.

### A. Thermal Cycling Conditions

Nucleic acid amplification and concurrent fluorescent signal detection are performed utilizing an automated real-time thermal cycling apparatus. To successfully execute the highly multiplexed diagnostic architectures described herein, the selected instrumentation must comprise a multi-well thermal block (e.g., a standard 96-well or 384-well format) capable of executing precise thermal ramping profiles, coupled with a multi-channel optical detection system. Specifically, the optical system must be configured to simultaneously excite and precisely resolve the emission spectra of at least four to six distinct fluorescent reporter moieties within a single reaction well, substantially without optical cross-talk.

The diagnostic methods and coordinated reaction sets of the present application are advantageously designed as an “open-system” architecture, ensuring robust analytical performance across a variety of commercially available real-time PCR platforms. By way of example, but not limitation, amplification and detection may be carried out on established clinical diagnostic platforms such as the CFX Opus 96 Real-Time PCR System (Bio-Rad Laboratories, Inc., Catalog No. 12011319), the QuantStudio™ 5 Real-Time PCR System (Thermo Fisher Scientific, Inc., Catalog No. A34322), or the SLAN-96S Real-Time PCR System (Shanghai Hongshi Medical Technology Co., Ltd.). The compatibility with such diverse, clinically validated instrumentation ensures broad clinical utility and facilitates seamless integration into standard molecular pathology laboratory workflows.

The highly multiplexed real-time polymerase chain reaction (qPCR) assays of the present application are executed under rigorously defined thermal cycling conditions. To achieve optimal target amplification while strictly preventing non-specific primer binding and primer-dimer formation, the thermal cycling parameters are strategically calibrated. Broadly, the amplification protocol comprises an optional initial enzymatic decontamination step (ranging from about 30° C. to 45° C. for 1 to 5 minutes), an initial denaturation/enzyme activation step (ranging from about 90° C. to 98° C. for 10 seconds to 5 minutes), followed by a plurality of amplification cycles (ranging from 35 to 55 cycles). Each amplification cycle broadly comprises a denaturation phase (ranging from about 90° C. to 98° C. for 1 to 30 seconds) and a combined annealing, extension, and fluorescence acquisition phase (ranging from about 55° C. to 65° C. for 15 to 60 seconds).

In highly optimized, exemplary embodiments, the specific thermal profiles are tailored to the biochemical kinetics of the selected master mix formulation. For example, when employing a hot-start formulation such as the QuantiNova® PCR Master Mix (Qiagen), the preferred thermal cycling protocol comprises an initial enzyme activation step at about 95° C. for 2 minutes, followed by 40 to 50 amplification cycles (preferably exactly 45 cycles). Each cycle optimally comprises a denaturation step at 95° C. for 5 seconds and a combined annealing/extension/acquisition step at 60° C. for 30 seconds.

Alternatively, when employing a formulation equipped with an enzymatic anti-contamination system, such as the BioSmart™ U+ 2× All-Powerful qPCR PreMix (Vazyme), the preferred thermal cycling protocol incorporates a preliminary Uracil-DNA Glycosylase (UDG)-mediated decontamination step at about 37° C. for 2 minutes. This is followed by an initial denaturation and UDG-inactivation step at about 95° C. for 30 seconds, and subsequently 40 to 50 amplification cycles (preferably exactly 45 cycles). Each cycle optimally comprises a denaturation step at 95° C. for 10 seconds and a combined annealing/extension/acquisition step at 60° C. for 30 seconds.

Furthermore, the thermodynamic precision of the multiplex assay is heavily dependent on specific thermal ramp rates, which control the transition kinetics between the denaturation and annealing phases. While ramp rates are inherently instrument-specific, they are strictly calibrated in the present application to maximize specific hybridization. In preferred embodiments, the thermal ramp rate is configured to be approximately 3.3° C. per second when utilizing the CFX Opus 96 system, approximately 3.4° C. per second when utilizing the QuantStudio™ 5 system, and approximately 4.0° C. per second when utilizing the SLAN-96S system.

The foregoing thermal cycling profiles and instrument-specific ramp rates demonstrate the highly optimized operability of the disclosed methods. However, it is expressly understood that comparable, functionally equivalent thermal cycling parameters—falling within the broader disclosed ranges—may be readily adapted and employed on other real-time PCR instrumentation capable of multiplex fluorescence detection without departing from the spirit and scope of the claimed application.

### B. Multiplex Reaction Setup

To ensure reproducible amplification kinetics and meet the stringent diagnostic standards required for clinical regulatory approval (such as NMPA Class III IVD registration), the multiplex amplification reactions are prepared within strictly calibrated reaction volumes. Broadly, the final reaction volume for each discrete multiplex pool may range from about 10 μL to about 50 μL. More preferably, the total volume ranges from about 15 μL to about 25 μL.

In highly optimized, preferred embodiments, each discrete multiplex reaction is precisely assembled in a final volume of approximately 15 μL. This optimized volume comprises: a high-fidelity PCR master mix (incorporating a thermostable DNA polymerase, deoxynucleotides, optimized buffer components, and necessary stabilizers); a strategically pooled mixture of the viral loci-specific primers and fluorescent probes; and the extracted nucleic acid template derived from the biological sample.

The volumetric ratio of the reaction components is critically balanced to prevent reagent exhaustion while maximizing the fluorescent signal-to-noise ratio in a highly multiplexed environment. In broadly defined configurations, the reaction mixture comprises approximately 30% to 50% by volume of a 2×PCR master mix, approximately 30% to 50% by volume of the pooled oligonucleotide mixture, and approximately 10% to 30% by volume of the nucleic acid template.

In the most preferred, highly calibrated 15 μL embodiment, the reaction precisely comprises about 6 μL of the 2×PCR master mix, about 6 μL of the pooled primers and probes, and about 3 μL of the nucleic acid template. As previously established, the amplification reagents preferably possess advanced enzymatic features (e.g., hot-start mechanisms and UDG-mediated decontamination), which may be sourced from commercial formulations such as the QuantiNova® Probe PCR Kit (Qiagen) or the BioSmart™ U+ All-Powerful Multiple Probe qPCR PreMix (Vazyme), among other functionally equivalent, clinical-grade reagents.

The physical assembly of the assay is conducted within reaction vessels that are optically and thermodynamically compatible with automated real-time PCR instrumentation. While standard high-throughput formats such as 96-well or 384-well plates are utilized, the reactions are specifically organized into the aforementioned “coordinated reaction set” (e.g., an 8-well strip configuration) to systematically distribute the comprehensive HPV target panel and the HBB internal control.

The formulated reaction plates are configured for immediate optical compatibility with validated, multi-channel multiplex detection systems. Representative clinical instruments include, but are not limited to, the CFX Opus 96 Real-Time PCR System (Bio-Rad Laboratories, Inc.), the QuantStudio™ 5 Real-Time PCR System (Thermo Fisher Scientific, Inc.), and the SLAN-96S Real-Time PCR System (Shanghai Hongshi Medical Technology Co., Ltd.). This structural and hardware versatility ensures the assay can be seamlessly integrated into diverse molecular pathology laboratory workflows without departing from the scope of the claimed multiplex architecture.

### C. Detection and Genotyping of HPVs

Fluorescent signal assignment is strictly configured to enable the simultaneous detection and clinical differentiation of multiple HPV genotypes within the highly multiplexed amplification reactions. In preferred embodiments, each specific HPV genotype, or defined clinical risk group of genotypes, is associated with a distinct fluorophore-quencher combination. This strategic optical arrangement permits the precise optical isolation and unambiguous identification of individual viral targets—as well as the universally integrated HBB internal control—based on their respective, non-overlapping fluorescence emission profiles, thereby actively preventing spectral cross-talk within any single reaction vessel.

To facilitate this extreme level of multiplexing and to absolutely minimize biochemical signal interference, the comprehensive panel of HPV targets is systematically organized into a coordinated plurality of discrete multiplex qPCR reaction pools. To accommodate varying clinical panel sizes and instrumentation limits, the reaction set broadly comprises from about 4 to about 16 discrete multiplex pools. More preferably, the reaction set comprises from about 6 to about 10 discrete pools. In the highly optimized, exemplary embodiment for the 43-genotype panel, the targets are physically distributed across exactly eight multiplex qPCR reaction pools.

Crucially, the segregation of targets across these eight multiplex pools is structurally engineered based on a synthesized algorithmic weighting of clinical and analytical factors. These grouping criteria explicitly include carcinogenic risk classification (e.g., separating High-Risk from Low-Risk types to streamline diagnostic reporting), analytical amplification efficiencies, and established epidemiological patterns of prevalent co-infections. By deliberately segregating frequently co-occurring HPV subtypes into completely separate physical reaction pools—or assigning them to distinctly separated optical channels within the same pool—the physical assay architecture actively circumvents polymerase competition. This structural optimization ensures the highly sensitive, independent detection of rare or lower-titer viral strains, even in complex, mixed-genotype clinical presentations.

Representative combinatorial mappings of the specific HPV genotypes to their designated fluorophore-quencher pairs, as well as their optimized spatial assignments within the 8-pool multiplex reaction set, are detailed in FIG. 2. Such mappings are illustrative of the preferred clinical architecture and are not intended to limit the broader scope of the disclosed diagnostic systems, methods, or biochemical compositions.

### 4. Analytical and Clinical Validation

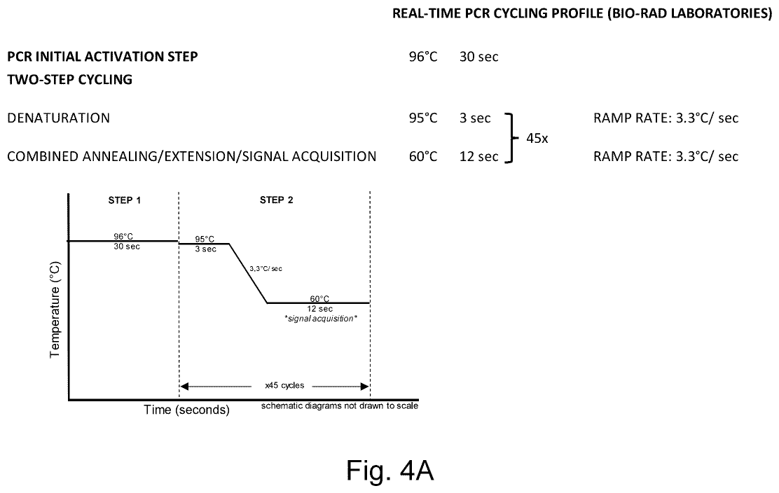

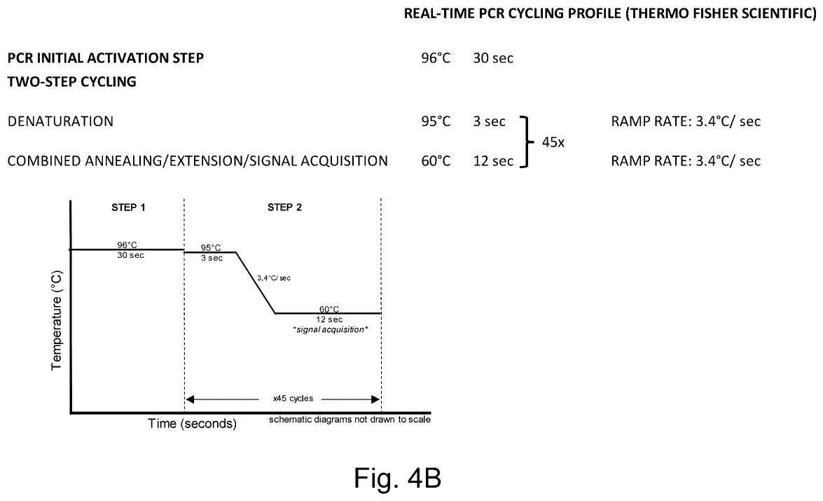

To satisfy the rigorous performance standards required for high-complexity in vitro diagnostic (IVD) regulatory registration, the multiplex diagnostic architectures described herein were subjected to extensive analytical optimization and large-scale clinical validation. As detailed in the supplementary figures, the assay parameters—including the precise thermal cycling profiles (e.g., FIGS. 4A and 4B) and the strictly calibrated concentration ratios of the oligonucleotides and dual-labeled probes (e.g., FIGS. 5A and 5B)—were empirically optimized across multiple established clinical platforms (including Thermo Fisher Scientific and Bio-Rad Laboratories instrumentation). These highly optimized parameters ensure maximum amplification efficiency and entirely prevent spectral cross-talk within the coordinated reaction sets. The robustness of this optimized formulation was subsequently clinically validated using a high-throughput cohort comprising thousands of biological cervical specimens.

The diagnostic sensitivity and analytical specificity of the disclosed multiplex assay were rigorously evaluated through large-scale clinical concordance studies. The assay's performance was benchmarked against orthogonal, gold-standard genomic methodologies (e.g., Sanger sequencing) as well as established, regulatory-approved legacy reference diagnostics (such as the Hybribio 37 HPV GenoArray Diagnostic Kit and the Roche Linear Array HPV Genotyping Test). In these comprehensive clinical evaluations, the disclosed 43-genotype multiplex assay demonstrated unparalleled diagnostic accuracy, achieving a cumulative clinical sensitivity of 100% and a cumulative clinical specificity of 99.7%. This exceptional performance actively demonstrates the assay's ability to completely circumvent false negatives associated with viral integration (via synergistic E4/E6 targeting) while maintaining strict sequence specificity to prevent false positives.

In summary, the present application provides a highly advanced, structurally engineered methodology and associated diagnostic systems for the comprehensive detection and precise clinical differentiation of 43 distinct HPV genotypes. By systematically distributing these targets across exactly eight discrete multiplex qPCR reaction pools—and strategically allocating them based upon established carcinogenicity, prevalence, and historical co-infection competition models—the disclosed architecture overcomes the multiplexing limitations of standard PCR instrumentation. Consequently, the application provides an incredibly robust, high-fidelity diagnostic tool capable of delivering immediate, risk-stratified clinical genotyping from a single patient specimen.

### EXAMPLES

### Example 1—Materials and Methods

### A. Oligonucleotide Synthesis and Probe Labeling

The viral gene loci-specific primers and sequence-specific dual-labeled fluorescent DNA probes of the present application (comprising SEQ ID NOs: 1 through 168) were custom-designed by the inventors to strategically target the L1, E4, and E6 viral gene loci, as well as the endogenous human beta-globin (HBB) internal control (SEQ ID NOs: 169-171). The synthesis of these oligonucleotide sequences is accomplished utilizing industry-standard automated DNA synthesis techniques conventionally known to those skilled in the art.

All of the probes are labeled using routine techniques known in the art. The fluorophores (e.g., FAM, HEX, ROX) are covalently attached to the 5′ end during synthesis or via post-synthetic conjugation, and the quenchers (e.g., BHQ1, BHQ2) are incorporated at the 3′ end using modified controlled pore glass (CPG) solid supports. The synthesized oligos are then purified via HPLC to ensure clinical-grade quality.

### B. Sample Preparation and Nucleic Acid Extraction

Biological specimens, such as cervical exfoliated cells, were collected utilizing standard sterile cytobrushes and immediately suspended in a clinically validated liquid-based cytology transport medium (e.g., PreservCyt® or a functionally equivalent viral transport medium) to preserve cellular and nucleic acid integrity.

Total nucleic acids (including viral episomal and integrated host genomic DNA) were subsequently extracted from an aliquot of the transport medium (e.g., 200 μL to 1 mL). Extraction was performed utilizing a standardized, automated magnetic bead-based viral DNA extraction system (or equivalent silica-column-based method) according to the manufacturer's validated protocols. The extracted, highly purified nucleic acids were eluted in a standardized elution buffer (e.g., 50 μL to 100 μL) and either immediately subjected to the highly multiplexed qPCR amplification reactions or stored at −20° C. or −80° C. to maintain stability prior to testing.

### C. Real-Time PCR

Nucleic acid amplification was carried out on a CFX Opus 96 Real-Time PCR System (Bio-Rad Laboratories, Catalog No. 12011319) under defined thermal cycling conditions.

For reactions employing QuantiNova PCR Master Mix (Qiagen), the thermal cycling protocol comprised an initial enzyme activation step at 95° C. for 2 minutes, followed by 45 amplification cycles. Each amplification cycle included a denaturation step at 95° C. for 5 seconds and a combined annealing, extension, and fluorescence signal acquisition step at 60° C. for 30 seconds, during which fluorescence data were collected.

For reactions employing BioSmart U+ 2× All-Powerful qPCR PreMix (Vazyme), the thermal cycling protocol comprised a preliminary Uracil-DNA Glycosylase (UDG)-mediated decontamination step at 37° C. for 2 minutes, followed by an initial denaturation and UDG-inactivation step at 95° C. for 30 seconds, and then 45 amplification cycles. Each amplification cycle included a denaturation step at 95° C. for 10 seconds and a combined annealing, extension, and fluorescence signal acquisition step at 60° C. for 30 seconds, during which fluorescence data were collected.

The ramp rate was set to approximately 3.3° C. per second when amplification was performed on the CFX Opus 96 Real-Time PCR System.

### Example 2—Sample Test and Data Analysis

In this example, 1789 samples containing HPV DNA targets were tested using the materials and methods described in Example 1. Some representative samples are extracted for the following analysis.

### 1. Representative Clinical Dataset Demonstrating Analytical Performance of the Multiplex HPV Assay

A representative selection of clinical specimens (n≈30) was assembled from the database to demonstrate the analytical performance characteristics of the disclosed high-multiplex HPV quantitative PCR assay. The selected specimens encompass a spectrum of clinically relevant diagnostic states and were chosen to illustrate assay behavior across multiple HPV genotypes, specimen types, and reaction chemistries.

The dataset includes (i) single-genotype high-risk HPV infections, (ii) single-genotype low-risk HPV infections, (iii) co-infections involving three to five HPV genotypes, (iv) complex co-infections involving six or more HPV genotypes, and (v) HPV-negative specimens. This structured selection enables demonstration of assay specificity, multiplex discrimination capability, and robustness under conditions of high target diversity.

Across the dataset, specimens were obtained from ThinPrep cervical specimens, swabs, and urine samples. Each sample was amplified using either the Qiagen or Vazyme PCR master mix, and all assays were performed on the CFX Opus real-time PCR instrument under standardized thermal cycling conditions. For each specimen, the table reports the detected HPV genotype(s), the corresponding Ct values at an RFU threshold of 200, the total number of genotypes present, and internal control performance.

This representative 43-genotype HPV panel demonstrates that the disclosed assay consistently and accurately identifies HPV genotypes across a wide range of clinical sample types and infection complexities. In particular, in complex co-infections (≥6 HPV genotypes), the assay maintains clear genotypic resolution without evidence of signal suppression or template competition, thereby supporting the function of the disclosed 8-pool architecture for multiplex discrimination. Negative specimens further confirm the absence of non-specific amplification and the reliability of internal controls.

TABLE 1
Representative 43-genotype HPV Panel

| | | | PCR Master | | 43-genotype HPV | | | | | | | | | | | | |
|---|---|---|---|---|---|---|---|---|---|---|---|---|---|---|---|---|---|
| Date | Case ID | Specimen Type | Mix | Instrument | Panel Result | Category | Ct values of HBB and Detected HPV Genotypes (RFU threshold: 200) | | | | | | | | | | n_types |
| 3 Aug. 2021 | HPV37-01 | Urine | Qiagen | CFX Opus | HPV18 POS | Single HRT | HBB | 18 | | | | | | | | | 1 |
| | | | | | | | 26.19 | 31.40 | | | | | | | | | |
| 12 Apr. 2025 | DR1 | THINPAP | Qiagen | CFX Opus | HPV52 POS | Single HRT | HBB | 52 | | | | | | | | | 1 |
| | | | | | | | 25.24 | 27.97 | | | | | | | | | |
| 28 May 2025 | DR5 | THINPAP | Qiagen | CFX Opus | HPV39 POS | Single HRT | HBB | 39 | | | | | | | | | 1 |
| | | | | | | | 25.26 | 28.66 | | | | | | | | | |
| 23 Oct. 2025 | DR9 | THINPAP | Qiagen | CFX Opus | HPV52 POS | Single HRT | HBB | 52 | | | | | | | | | 1 |
| | | | | | | | 25 | 27.02 | | | | | | | | | |
| 12 Nov. 2025 | DR19 | THINPAP | Qiagen | CFX Opus | HPV33 POS | Single HRT | HBB | 33 | | | | | | | | | 1 |
| | | | | | | | 24.23 | 25.99 | | | | | | | | | |
| 17 Jan. 2026 | DR9 | THINPAP | Vazyme | CFX Opus | HPV51 POS | Single HRT | HBB | 51 | | | | | | | | | 1 |
| | | | | | | | 27.77 | 34.37 | | | | | | | | | |
| 7 Apr. 2025 | DR5 | THINPAP | Qiagen | CFX Opus | HPV54 POS | Single LRT | HBB | 54 | | | | | | | | | 1 |
| | | | | | | | 26.77 | 37.78 | | | | | | | | | |
| 13 Oct. 2025 | DR6 | THINPAP | Qiagen | CFX Opus | HPV54 POS | Single LRT | HBB | 54 | | | | | | | | | 1 |
| | | | | | | | 25.03 | 30.53 | | | | | | | | | |
| 13 Oct. 2025 | DR7 (1008-DR14) | THINPAP | Qiagen | CFX Opus | HPV90 POS | Single LRT | HBB | 90 | | | | | | | | | 1 |
| | | | | | | | 29.28 | 27.16 | | | | | | | | | |
| 4 Nov. 2025 | DR13 | THINPAP | Qiagen | CFX Opus | HPV40 POS | Single LRT | HBB | 40 | | | | | | | | | 1 |
| | | | | | | | 28.05 | 42.21 | | | | | | | | | |
| 12 Jan. 2026 | BCHK1020 | Urine | Vazyme | CFX Opus | HPV6 POS | Single LRT | HBB | 6 | | | | | | | | | 1 |
| | | | | | | | 30.06 | 37.11 | | | | | | | | | |
| 17 Jan. 2026 | DR8 | THINPAP | Vazyme | CFX Opus | HPV54 POS | Single LRT | HBB | 54 | | | | | | | | | 1 |
| | | | | | | | 24.63 | 35.15 | | | | | | | | | |
| 19 Jul. 2025 | DR2 | THINPAP | Qiagen | CFX Opus | HPV42, 52, 73 POS | Co-infection | HBB | 42 | 52 | 73 | | | | | | | 3 |
| | | | | | | (3-5) | 24.42 | 22.00 | 23.2 | 20.54 | | | | | | | |
| 9 Dec. 2025 | DR12 | THINPAP | Qiagen | CFX Opus | HPV42, 52, 58 POS | Co-infection | HBB | 42 | 52 | 58 | | | | | | | 3 |
| | | | | | | (3-5) | 25.49 | 37.60 | 18.15 | 15.47 | | | | | | | |
| 13 Jan. 2026 | DR14 | Swab | Vazyme | CFX Opus | HPV31, 39, 56, 68 POS | Co-infection | HBB | 31 | 39 | 56 | 68 | | | | | | 4 |
| | | | | | | (3-5) | 23.18 | 23.24 | 16.01 | 13.32 | 19.23 | | | | | | |
| 19 Jan. 2026 | DR15 | THINPAP | Vazyme | CFX Opus | HPV 51, 62, 89 POS | Co-infection | HBB | 51 | 62 | 89 | | | | | | | 3 |
| | | | | | | (3-5) | 23.14 | 26.27 | 28.05 | 36 | | | | | | | |
| 10 Aug. 2018 | HPV37-01 | Swab | Qiagen | CFX Opus | HPV16, 26, 68, 81, 84, | Complex co- | HBB | 16 | 26 | 68 | 81 | 84 | 89 | | | | 6 |
| | | | | | 89 POS | infection (≥6) | 24.39 | 34.29 | 32.15 | 17.07 | 24.83 | 29.78 | 26.07 | | | | |
| 18 Jan. 2019 | HPV37-01 | Swab | Qiagen | CFX Opus | HPV42, 51, 52, 54, 84, | Complex co- | HBB | 42 | 51 | 52 | 54 | 84 | 89 | | | | 6 |
| | | | | | 89 POS | infection (≥6) | 22.78 | 27.74 | 22.93 | 32.2 | 26.46 | 30.91 | 23.76 | | | | |
| 14 Feb. 2019 | HPV37-01 | Swab | Qiagen | CFX Opus | HPV42, 44, 51, 52, 55, | Complex co- | HBB | 42 | 44 | 51 | 52 | 55 | 58 | 59 | 66 | | 8 |
| | | | | | 58, 59, 66 POS | infection (≥6) | 23.03 | 33.00 | 22.67 | 31.21 | 18.12 | 22.55 | 15.96 | 15.3 | 23.7 | | |
| 23 Jan. 2020 | HPV37-02 | Swab | Qiagen | CFX Opus | HPV16, 34, 42, 51, 53, | Complex co- | HBB | 16 | 34 | 42 | 51 | 53 | 62 | | | | 6 |
| | | | | | 62 POS | infection (≥6) | 26.25 | 28.21 | 27.21 | 28.2 | 23.06 | 20.07 | 31.29 | | | | |
| 6 Aug. 2025 | DR13 | THINPAP | Qiagen | CFX Opus | HPV26, 39, 52, 56, 62, | Complex co- | HBB | 26 | 39 | 52 | 56 | 62 | 68 | 81 | 89 | | 8 |
| | | | | | 68, 81, 89 POS | infection (≥6) | 25.63 | 26.31 | 30.25 | 27.78 | 33.02 | 26.96 | 26.12 | 24.6 | 37.5 | | |
| 21 Jan. 2026 | DR4 | Swab | Vazyme | CFX Opus | HPV39, 42, 51, 52, 53, | Complex co- | HBB | 39 | 42 | 51 | 52 | 53 | 54 | 58 | 62 | 82 | 9 |
| | | | | | 54, 58, 62, 82 POS | infection (≥6) | 23.7 | 38.18 | 28.67 | 23.47 | 35.62 | 34.59 | 26.36 | 30.3 | 24.4 | 36.1 | |
| 3 Jan. 2019 | HPV37-01 | Urine | Qiagen | CFX Opus | ALL NEG | Negative | HBB | | | | | | | | | | 0 |
| | | | | | | | 32.28 | | | | | | | | | | |
| 12 Feb. 2019 | HPV37-01 | Urine | Qiagen | CFX Opus | ALL NEG | Negative | HBB | | | | | | | | | | 0 |
| | | | | | | | 32.29 | | | | | | | | | | |
| 31 Oct. 2019 | HPV37-03 | Urine | Qiagen | CFX Opus | ALL NEG | Negative | HBB | | | | | | | | | | 0 |
| | | | | | | | 29.03 | | | | | | | | | | |
| 14 Jan. 2026 | DR19 | THINPAP | Vazyme | CFX Opus | ALL NEG | Negative | HBB | | | | | | | | | | 0 |
| | | | | | | | 27.19 | | | | | | | | | | |
| 15 Jan. 2026 | DA12 | Urine | Vazyme | CFX Opus | ALL NEG | Negative | HBB | | | | | | | | | | 0 |
| | | | | | | | 33.03 | | | | | | | | | | |
| 15 Jan. 2026 | DR6 | Swab | Vazyme | CFX Opus | ALL NEG | Negative | HBB | | | | | | | | | | 0 |
| | | | | | | | 25.81 | | | | | | | | | | |

### 2. Fail-Safe Detection Mechanism Evidence Table

To validate the fail-safe detection mechanism incorporated into the disclosed HPV qPCR system, a subset of HPV33-positive clinical specimens was analyzed with respect to differential amplification of the L1 and E4 genomic regions.

In this analysis, specimens were subjected to serial dilution (neat, 1:50, and 1:100) and amplified independently for HPV33 L1, HPV33 E4, and a combined L1+E4 configuration. Across multiple specimens, the L1 target frequently exhibited weak amplification or non-detectable signal at higher dilutions, whereas the E4 target remained detectable with robust amplification. In all cases, the combined L1+E4 assay correctly identified the samples as HPV33 positive. These data demonstrate that retention of E4 detection in the assay design provides a functional safeguard allowing accurate identification of HPV-positive specimens even when L1 integrity is compromised.

TABLE 2

| | | | | | Ct values @ DNA Dilutions | | | | | Fail-safe |
|---|---|---|---|---|---|---|---|---|---|---|
| | | Case | HPV | P/P gene | | 1:50 | 1:50 | 1:100 | 1:100 | evidence |
| Sample | Date | ID | target | Target | Neat | (Replicate 1) | (Replicate 2) | (Replicate 1) | (Replicate 2) | (L1−, E4+) |
| 2 | 12 Sep. 2025 | DR6 | HPV33 | L1 + E4 | 27.59 | 33.61 | 34.26 | 37.01 | 34.57 | Yes |
| | | | | L1 | 28.30 | ND | ND | ND | ND | |
| | | | | E4 | 28.51 | 33.61 | 33.64 | 35.87 | 36.01 | |
| 3 | 1 Dec. 2025 | DR11 | HPV33 | L1 + E4 | 27.05 | 34.38 | 33.83 | 33.77 | 34.59 | Yes |
| | | | | L1 | 28.05 | ND | ND | ND | ND | |
| | | | | E4 | 28.11 | 33.04 | 32.94 | 34.12 | 35.00 | |
| 4 | 17 Jan. 2026 | DR5 | HPV33 | L1 + E4 | L1 + E4 | 29.84 | 38.94 | 39.52 | 40.99 | Yes |
| | | | | L1 | 31.16 | ND | ND | ND | ND | |
| | | | | E4 | 30.91 | 35.23 | 35.50 | 44.20 | 35.91 | |

ND: Not Detected

Accordingly, the results support the utility and reliability of the disclosed dual-target architecture in ensuring diagnostic accuracy under clinically relevant integration conditions.

### 3. Comparative Study

A comparative study was conducted to evaluate the analytical performance of an immunoblot-based HPV genotyping assay (Hybribio 37 HPV GenoArray Diagnostic Kit) relative to a multiplex quantitative PCR assay capable of detecting 43 HPV genotypes (“43-genotype HPV panel”). Matching clinical specimens were subjected to both testing methods. Immunoblot images were interpreted according to the manufacturer's membrane-banding criteria, while the 43-genotype HPV panel provided genotype-specific positive calls supported by corresponding cycle threshold (Ct) values.

A curated subset of representative samples (n=10) was selected from the full dataset. These cases were chosen to highlight the most demonstrative scenarios for comparison, particularly those that: (i) show instances in which the Hybribio assay detected fewer genotypes than the 43-genotype HPV Panel in multi-genotype infections; (ii) show instances in which the Hybribio assay failed to detect certain high-risk carcinogenic (HRT) subtypes; and (iii) demonstrate the substantially broader genotype coverage provided by the 43-genotype HPV Panel.

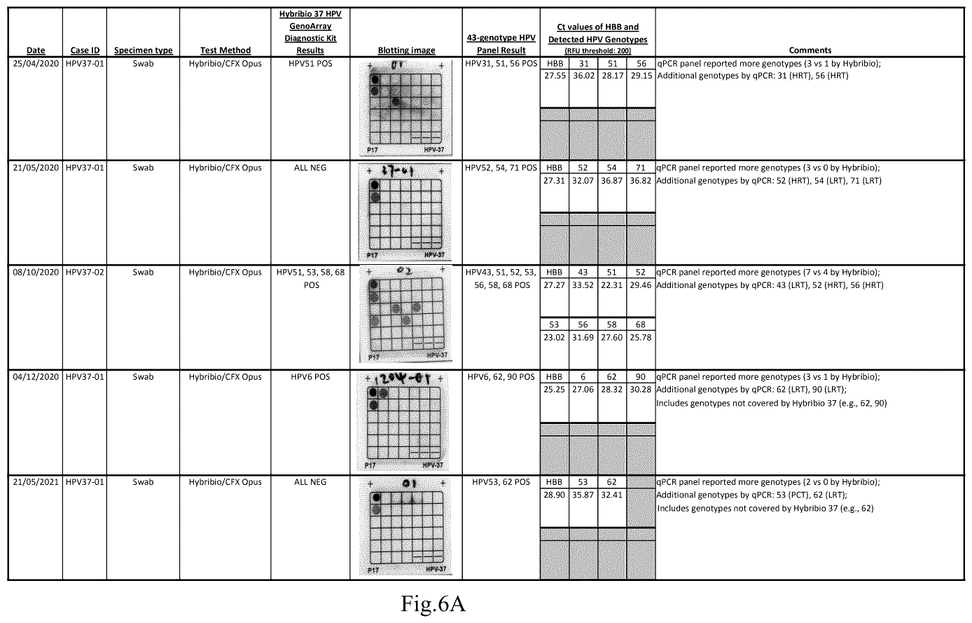

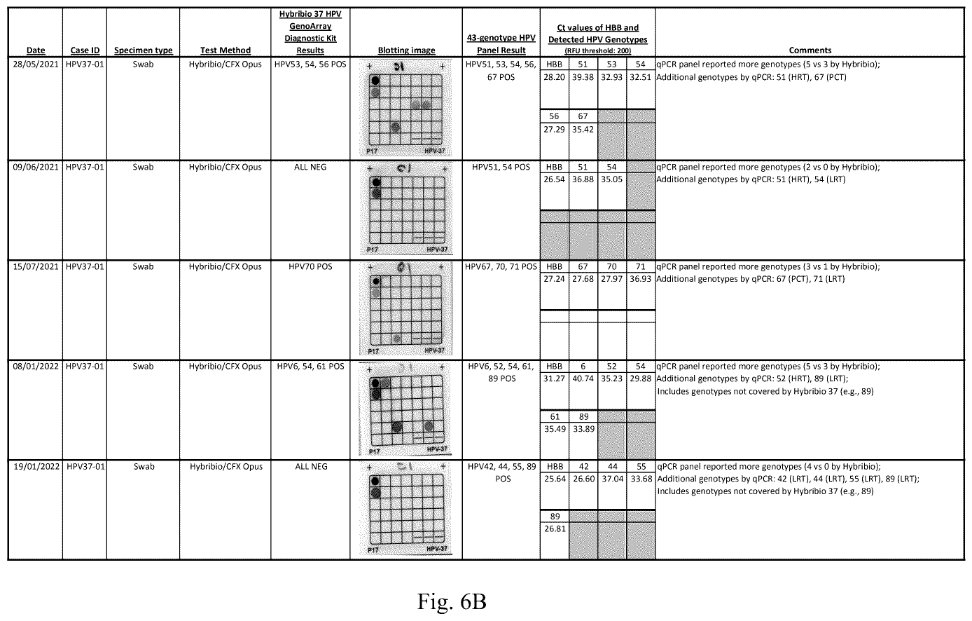

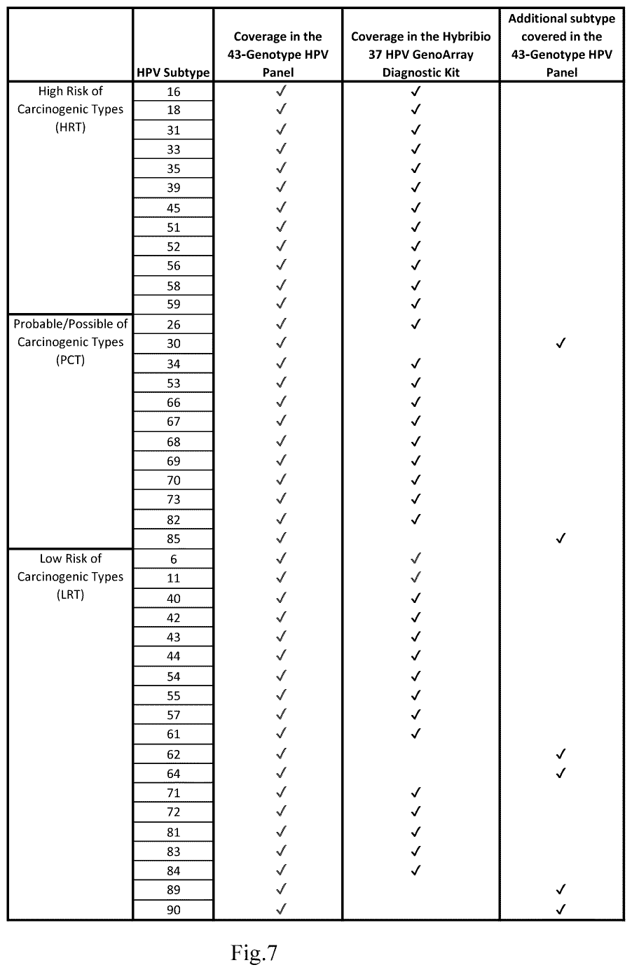

To support clearer understanding, the corresponding data tables are provided in FIGS. 6A, 6B and 7.

In FIGS. 6A and 6B, the table summarizes for each case, the Hybribio immunoblot result, the corresponding blot image, the 43-genotype HPV panel Ct values for all detected genotypes, and commentary on assay-specific discrepancies.

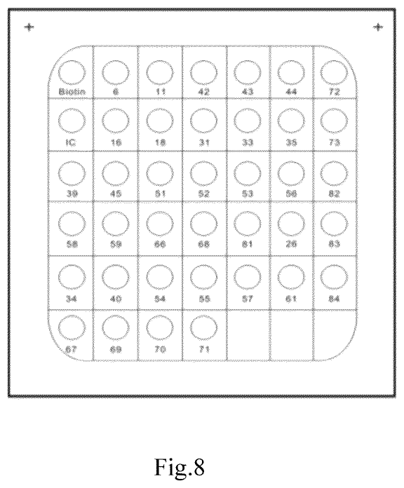

In FIG. 7, the table summarizes the genotypes detectable by the Hybribio 37-type assay versus those detectable by the 43-genotype HPV panel, making it straightforward to identify genotypes present only in the 43-genotype panel (e.g., HPV30, HPV62, HPV64, HPV85, HPV89, HPV90). The diagram in FIG. 8 illustrates the location of specific HPV probes on the HybriMem membrane of the Hybribio 37 HPV GenoArray Diagnostic Kit, in which Biotin control (Biotin) is located at the upper left-handed corner and it is to monitor the whole process of hybridization; and Internal control (IC) is located under the Biotin control, and is to monitor for the efficiency of PCR amplification, presence of PCR inhibition and reagent integrity. For the results interpretation, the presence of both Biotin and IC controls=Normal Result, True Negative; and the presence of all Biotin, IC controls and any HPV types=Normal Result, True Positive.

Across the dataset, the 43-genotype HPV panel consistently identified a greater number of HPV genotypes per specimen when compared with the immunoblot. In many instances, the immunoblot reported either a reduced number of detectable HPV types or a fully negative result, whereas the 43-genotype HPV panel detected multiple HPV genotypes with Ct values falling within the expected positive detection range. For example, cases dated 25 Apr. 2020, 21/05/2020 and Sep. 6, 2021 each demonstrate scenarios in which Hybribio method reported either a single genotype or a complete absence of detectable HPV, while the 43-genotype HPV panel identified two or more genotypes within the same specimen. These additional qPCR-identified genotypes were supported by Ct values within the mid-20s to mid-30s, confirming reliable detection rather than borderline amplification.

Notably, in multiple representative cases, for example, cases dated 25 Apr. 2020, 21/05/2020, Aug. 10, 2020, 28/05/2021, Sep. 6, 2021 and Aug. 1, 2021, the Hybribio assay failed to detect HRT subtypes (e.g. HPV31, HPV51, HPV52, HPV56) despite the 43-genotype HPV panel identifying these genotypes with Ct values well within the expected positive detection range.

Moreover, cases involving multi-genotype infections clearly illustrate performance limitations of the immunoblot platform. In specimens such as those dated Aug. 10, 2020, 28/05/2021, and 15/07/2021, the 43-genotype HPV panel detected complex co-infection patterns comprising 4-7 distinct HPV genotypes, whereas the immunoblot displayed incomplete or weak hybridization bands for several of these types. The absence or faintness of membrane signals for these genotypes reflects the reduced hybridization sensitivity of the immunoblot, particularly when multiple HPV types are present at varying abundance levels.

In addition, the 43-genotype HPV panel detected several HPV types not included in the Hybribio assay menu. These include HPV30, HPV62, HPV64, HPV85, HPV89, and HPV90, which were identified in specimens dated Apr. 12, 2020, 21/05/2021, Aug. 1, 2022 and 19/01/2022, among others. In these cases, the immunoblot either returned a negative result or detected only one to two HPV genotypes, while the 43-genotype HPV panel demonstrated broader coverage.

Collectively, these comparative data demonstrate that the 43-genotype HPV panel provides better analytical sensitivity, enhanced detection of multi-genotype co-infections, and substantially broader genotype coverage when compared with the Hybribio immunoblot assay. The 43-genotype HPV panel reduces genotype dropouts and false-negative results commonly associated with membrane-based hybridization, thereby offering a more comprehensive and accurate assessment of HPV genotype diversity within clinical specimens.

### Example 3—Clinical Performance of the Method

This example tests the sensitivity and specificity of the method through a parallel clinical concordance study. The clinical specimens were tested head-to-head using our novel 43-plex assay against the legacy reference assays (Hybribio and Roche).

Crucially, for any sample where our assay produced a discordant result compared to the legacy tests (e.g., we called it positive, but Roche called it negative), we did not automatically assume our test was wrong. Instead, we used bidirectional Sanger sequencing on those specific discordant samples as the ultimate ‘gold standard’ tie-breaker to determine the true clinical status of the sample.

Based on the finalized, Sanger sequencing-verified dataset comprising a total sample size of n=1789, standard epidemiological formulas were utilized to determine the diagnostic performance of the multiplex assay. Specifically, the clinical concordance analysis evaluated the incidence of true positives, false positives, true negatives, and false negatives to calculate a cumulative diagnostic sensitivity of 100% and a specificity of 99.7%. Furthermore, these analytical parameters were utilized to determine the Positive Predictive Value (PPV) and Negative Predictive Value (NPV), thereby demonstrating the high diagnostic accuracy and clinical reliability of the assay.

It should be understood that the foregoing detailed description and specific examples, while indicating preferred and exemplary embodiments of the present application, are given by way of illustration and not by way of limitation. It will be readily apparent to those skilled in the relevant art that various modifications, alterations, substitutions, and adaptations may be made to the described diagnostic methods, biochemical compositions, and multiplex architectures without departing from the fundamental spirit and scope of the application. All such equivalent modifications and variations are intended to be included within the scope of the present disclosure and the appended claims.

Furthermore, it should be appreciated that certain features of the application, which are, for clarity and ease of understanding, described in the context of separate, discrete embodiments, may also be provided in combination in a single, integrated embodiment. Conversely, various features of the application which are, for brevity, described in the context of a single embodiment or a unified diagnostic system, may also be provided separately, independently, or in any suitable subcombination. The present disclosure expressly contemplates and protects all such structural and methodological permutations.

All patents, patent applications, scientific publications, manufacturer protocols, and other references specifically cited or identified herein are hereby incorporated by reference in their entireties, to the same extent as if each individual publication or patent application was specifically and individually indicated to be incorporated by reference. However, the citation, discussion, or incorporation of such references is strictly for the purpose of providing contextual background and is not necessarily to be construed as an admission as to its appropriateness, citability, and/or availability as invalidating prior art against the patentability of the present application.

What is claimed is:

1. A kit for detecting and simultaneously genotyping a plurality of Human Papillomavirus (HPV) genotypes in a biological sample, comprising: a plurality of physically discrete reaction pools configured to collectively detect and differentiate said plurality of HPV genotypes; wherein each reaction pool comprises a formulated, non-overlapping subset of viral gene loci-specific oligonucleotide triplets, each triplet comprising a forward primer, a reverse primer, and a sequence-specific dual-labeled fluorescent DNA probe; wherein the oligonucleotide triplets within the kit are configured to specifically hybridize to and amplify a synergistic combination of viral genetic targets, said targets comprising a highly conserved L1 structural gene and at least one early viral gene selected from the group consisting of an E4 gene and an E6 gene, thereby mitigating false-negative detection associated with viral episomal integration; and wherein at least one of said reaction pools further comprises an internal control primer-probe set targeting an endogenous human housekeeping gene to verify sample adequacy and the absence of amplification inhibitors.

2. The kit of claim 1, wherein the plurality of physically discrete reaction pools consists of exactly 8 reaction pools, and wherein each reaction pool is configured to detect and genotype one of the following groups of HPV genotypes:
a first group of HPV genotypes: HPV genotypes 30, 33, 52, 62, 70, and 72;
a second group of HPV genotypes: HPV genotypes 11, 18, 51, 57, 64, 69, and 85;
a third group of HPV genotypes: HPV genotypes 31, 34, 42, 53, and 90;
a fourth group of HPV genotypes: HPV genotypes 40, 44, 45, 54, 55, and 89;
a fifth group of HPV genotypes: HPV genotypes 26, 56, 58, 71, and 84;
a sixth group of HPV genotypes: HPV genotypes 59, 61, 68, 73, and 83;
a seventh group of HPV genotypes: HPV genotypes 6, 39, 43, 66, and 82; and
an eighth group of HPV genotypes: HPV genotypes 16, 35, 67, and 81.

3. The kit of claim 1, wherein:
the forward primer comprises or consists of a nucleotide sequence selected from the group consisting of SEQ ID NOs: 1, 4, 7, 10, 13, 16, 19, 22, 25, 28, 31, 34, 37, 40, 43, 46, 49, 52, 55, 58, 61, 64, 67, 70, 73, 76, 79, 82, 85, 88, 91, 94, 97, 100, 103, 106, 109, 112, 115, 118, 121, 124, 127, 130, 133, 136, 139, 142, 145, 148, 151, 154, 157, 160, 163, and 166, or a sequence having at least 90%, 95%, 98%, or 99% sequence identity thereto;
the reverse primer comprises or consists of a nucleotide sequence selected from the group consisting of SEQ ID NOs: 2, 5, 8, 11, 14, 17, 20, 23, 26, 29, 32, 35, 38, 41, 44, 47, 50, 53, 56, 59, 62, 65, 68, 71, 74, 77, 80, 83, 86, 89, 92, 95, 98, 101, 104, 107, 110, 113, 116, 119, 122, 125, 128, 131, 134, 137, 140, 143, 146, 149, 152, 155, 158, 161, 164, and 167, or a sequence having at least 90%, 95%, 98%, or 99% sequence identity thereto; and
the DNA probe comprises or consists of a sequence selected from the group consisting of SEQ ID NOs: 3, 6, 9, 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, 162, 165, and 168, or a sequence having at least 90%, 95%, 98%, or 99% sequence identity thereto.

4. The kit of claim 1, wherein each reaction pool comprises one or more forward primers selected from one of the following groups:
a first group of forward primers, each comprising or consisting of a nucleotide sequence selected from the group consisting of SEQ ID NOs: 1, 4, 7, 10, 13, 16, 19, and 22, or a sequence having at least 90%, 95%, 98%, or 99% sequence identity thereto;
a second group of forward primers, each comprising, consisting essentially of, or consisting of a nucleotide sequence selected from the group consisting of SEQ ID NOs: 25, 28, 31, 34, 37, 40, 43, and 46, or a sequence having at least 90%, 95%, 98%, or 99% sequence identity thereto;
a third group of forward primers, each comprising or consisting of a nucleotide sequence selected from the group consisting of SEQ ID NOs: 49, 52, 55, 58, 61, 64, and 67, or a sequence having at least 90%, 95%, 98%, or 99% sequence identity thereto;
a fourth group of forward primers, each comprising, or consisting of a nucleotide sequence selected from the group consisting of SEQ ID NOs: 70, 73, 76, 79, 82, and 85, or a sequence having at least 90%, 95%, 98%, or 99% sequence identity thereto;
a fifth group of forward primers, each comprising or consisting of a nucleotide sequence selected from the group consisting of SEQ ID NOs: 88, 91, 94, 97, 100, 103, and 106, or a sequence having at least 90%, 95%, 98%, or 99% sequence identity thereto;
a sixth group of forward primers, each comprising or consisting of a nucleotide sequence selected from the group consisting of SEQ ID NOs: 109, 112, 115, 118, 121, 124, and 127, or a sequence having at least 90%, 95%, 98%, or 99% sequence identity thereto;
a seventh group of forward primers, each comprising or consisting of a nucleotide sequence selected from the group consisting of SEQ ID NOs: 130, 133, 136, 139, 142, 145, and 148, or a sequence having at least 90%, 95%, 98%, or 99% sequence identity thereto;
an eighth group of forward primers, each comprising or consisting of a nucleotide sequence selected from the group consisting of SEQ ID NOs: 151, 154, 157, 160, 163, and 166, or a sequence having at least 90%, 95%, 98%, or 99% sequence identity thereto.

5. The kit of claim 1, wherein each reaction pool comprises one or more reverse primers selected from one of the following groups:
a first group of reverse primers, each comprising or consisting of a nucleotide sequence selected from the group consisting of SEQ ID NOs: 2, 5, 8, 11, 14, 17, 20, and 23, or a sequence having at least 90%, 95%, 98%, or 99% sequence identity thereto;
a second group of reverse primers, each comprising or consisting of a nucleotide sequence selected from the group consisting of SEQ ID NOs: 26, 29, 32, 35, 38, 41, 44, and 47, or a sequence having at least 90%, 95%, 98%, or 99% sequence identity thereto;
a third group of reverse primers, each comprising or consisting of a nucleotide sequence selected from the group consisting of SEQ ID NOs: 50, 53, 56, 59, 62, 65, and 68, or a sequence having at least 90%, 95%, 98%, or 99% sequence identity thereto;
a fourth group of reverse primers, each comprising, or consisting of a nucleotide sequence selected from the group consisting of SEQ ID NOs: 71, 74, 77, 80, 83, and 86, or a sequence having at least 90%, 95%, 98%, or 99% sequence identity thereto;
a fifth group of reverse primers, each comprising or consisting of a nucleotide sequence selected from the group consisting of SEQ ID NOs: 89, 92, 95, 98, 101, 104, and 107, or a sequence having at least 90%, 95%, 98%, or 99% sequence identity thereto;
a sixth group of reverse primers, each comprising or consisting of a nucleotide sequence selected from the group consisting of SEQ ID NOs: 110, 113, 116, 119, 122, 125, and 128, or a sequence having at least 90%, 95%, 98%, or 99% sequence identity thereto;
a seventh group of reverse primers, each comprising or consisting of a nucleotide sequence selected from the group consisting of SEQ ID NOs: 131, 134, 137, 140, 143, 146, and 149, or a sequence having at least 90%, 95%, 98%, or 99% sequence identity thereto; and
an eighth group of reverse primers, each comprising or consisting of a nucleotide sequence selected from the group consisting of SEQ ID NOs: 152, 155, 158, 161, 164, and 167 or a sequence having at least 90%, 95%, 98%, or 99% sequence identity thereto.

6. The kit of claim 1, each reaction pool comprises one or more probes selected from one of the following groups:
a first group of probes, each comprising or consisting of a nucleotide sequence selected from the group consisting of SEQ ID NOs: 3, 6, 9, 12, 15, 18, 21, 24, or a sequence having at least 90%, 95%, 98%, or 99% sequence identity thereto;
a second group of probes, each comprising or consisting of a nucleotide sequence selected from the group consisting of SEQ ID NOs: 27, 30, 33, 36, 39, 42, 45, 48, or a sequence having at least 90%, 95%, 98%, or 99% sequence identity thereto;
a third group of probes, each comprising or consisting of a nucleotide sequence selected from the group consisting of SEQ ID NOs: 51, 54, 57, 60, 63, 66, 69, or a sequence having at least 90%, 95%, 98%, or 99% sequence identity thereto;
a fourth group of probes, each comprising or consisting of a nucleotide sequence selected from the group consisting of SEQ ID NOs: 72, 75, 78, 81, 84, 87, or a sequence having at least 90%, 95%, 98%, or 99% sequence identity thereto;
a fifth group of probes, each comprising or consisting of a nucleotide sequence selected from the group consisting of SEQ ID NOs: 90, 93, 96, 99, 102, 105, 108, or a sequence having at least 90%, 95%, 98%, or 99% sequence identity thereto;
a sixth group of probes, each comprising or consisting of a nucleotide sequence selected from the group consisting of SEQ ID NOs: 111, 114, 117, 120, 123, 126, 129, or a sequence having at least 90%, 95%, 98%, or 99% sequence identity thereto;
a seventh group of probes, each comprising or consisting of a nucleotide sequence selected from the group consisting of SEQ ID NOs: 132, 135, 138, 141, 144, 147, 150, or a sequence having at least 90%, 95%, 98%, or 99% sequence identity thereto; and
an eighth group of probes, each comprising or consisting of a nucleotide sequence selected from the group consisting of SEQ ID NOs: 153, 156, 159, 162, 165, and 168, or a sequence having at least 90%, 95%, 98%, or 99% sequence identity thereto.

7. The kit of claim 1, wherein the internal control primer-probe set comprises: —a pair of internal control forward and reverse primers for amplifying the endogenous human housekeeping gene; and
an internal control probe for detecting an amplification product of the endogenous human housekeeping gene,
wherein the endogenous human housekeeping gene is a human beta globin (HBB) gene, wherein the internal control forward primer comprises or consists of a nucleotide sequence SEQ ID No. 169, or a sequence having at least 90%, 95%, 98%, or 99% sequence identity thereto, the internal control reverse control forward primer comprises or consists of a nucleotide sequence SEQ ID No. 170, or a sequence having at least 90%, 95%, 98%, or 99% sequence identity thereto, the internal control probe comprises or consists of a nucleotide sequence SEQ ID No. 171, or a sequence having at least 90%, 95%, 98%, or 99% sequence identity thereto.

8. The kit of claim 1, wherein the kit further comprises a PCR master mix comprising a thermostable DNA polymerase, a comprehensive deoxynucleotide triphosphate (dNTP) mix, buffer components providing essential divalent cations, and reaction stabilizers.

9. The kit of claim 1, wherein the dual-labeled fluorescent DNA probes within the kit comprise fluorophore-quencher combinations selected from the group consisting of FAM-BHQ1, HEX-BHQ1, VIC-BHQ1, TAMRA-BHQ2, ROX-BHQ2, Texas Red-BHQ2, Cy5.5-BHQ2, Quasar 670-BHQ2, Cy5-BHQ2, and functionally equivalent combinations thereof, and are configured to generate distinct emission spectra without significant optical cross-talk.

10. A method for detecting and simultaneously genotyping Human Papillomaviruses (HPVs) in a biological sample within a single reaction or a coordinated reaction set, comprising the steps of:
(a) partitioning the biological sample into a coordinated reaction set comprising a plurality of physically discrete reaction pools from the kit of claim 1 and contacting the biological sample with the subset of viral gene loci-specific oligonucleotide triplets comprised in the reaction pools to form reaction mixtures, each triplet comprising a forward primer, a reverse primer, and a sequence-specific dual-labeled fluorescent DNA probe;
(b) subjecting the reaction mixtures in the plurality of physically discrete reaction pools simultaneously to a nucleic acid amplification reaction under a PCR thermal cycling profile to generate target-specific amplicons;
(c) simultaneously detecting fluorescent optical signals emitted across multiple distinct detection channels within each of the reaction pools during the amplification reaction, thereby identifying and differentiating the HPV genotypes present in the biological sample.

11. The method of claim 10, wherein the biological sample is partitioned into exactly 8 reaction pools, and each reaction pool is configured to detect and genotype one of the following groups of HPV genotypes:
a first group of HPV genotypes: HPV genotypes 30, 33, 52, 62, 70, and 72;
a second group of HPV genotypes: HPV genotypes 11, 18, 51, 57, 64, 69, and 85;
a third group of HPV genotypes: HPV genotypes 31, 34, 42, 53, and 90;
a fourth group of HPV genotypes: HPV genotypes 40, 44, 45, 54, 55, and 89;
a fifth group of HPV genotypes: HPV genotypes 26, 56, 58, 71, and 84;
a sixth group of HPV genotypes: HPV genotypes 59, 61, 68, 73, and 83;
a seventh group of HPV genotypes: HPV genotypes 6, 39, 43, 66, and 82; and
an eighth group of HPV genotypes: HPV genotypes 16, 35, 67, and 81.

12. The method of claim 10, wherein the first to the eighth group of HPV genotypes are systematically organized based upon established clinical risk classifications and epidemiological co-infection prevalence.

13. The method of claim 10, wherein the biological sample is partitioned into eight reaction pools, and each of the eighth reaction pools comprises a group of probes specific for each of the first to the eighth group of HPV genotypes.

14. The method of claim 10, wherein the coordinated reaction set is configured to simultaneously detect and clinically differentiate up to 43 distinct HPV genotypes from the single biological sample.

15. The method of claim 10, wherein the viral gene loci-specific oligonucleotide triplets are engineered to specifically hybridize with and amplify a synergistic combination of viral genetic targets, specifically comprising the highly conserved structural L1 gene and the early oncogenic E6 gene, alongside the selective incorporation of the early E4 gene, thereby circumventing false-negative detection associated with viral episomal integration.

16. The method of claim 10, further comprising the concurrent amplification and optical detection of an endogenous assay internal control targeting the human beta-globin (HBB) gene, wherein oligonucleotides targeting said HBB gene are incorporated in at least one of the reaction pools to independently verify sample adequacy and the absence of PCR inhibitors on a reaction-by-reaction basis.

17. The method of claim 10, wherein the dual-labeled fluorescent DNA probes within the kit comprise fluorophore-quencher combinations selected from the group consisting of FAM-BHQ1, HEX-BHQ1, VIC-BHQ1, TAMRA-BHQ2, ROX-BHQ2, Texas Red-BHQ2, Cy5.5-BHQ2, Quasar 670-BHQ2, Cy5-BHQ2, and functionally equivalent combinations thereof, and are configured to generate distinct emission spectra without significant optical cross-talk.

18. The method of claim 10, wherein the kit comprises a PCR master mix, which comprises a thermostable hot-start DNA polymerase, an optimized PCR reaction buffer containing divalent cations, a comprehensive deoxynucleotide triphosphate (dNTP) mixture comprising dUTP, and a Uracil-DNA Glycosylase (UDG) enzyme for carryover contamination prevention.

19. The method of claim 10, wherein the probes hybridize to complementary sequences within the amplicons during step (b).

20. An automated diagnostic system for molecular genotyping of HPVs comprising:
(a) the kit of claim 1;
(b) a multi-channel real-time thermal cycling apparatus configured to execute the defined thermal cycling profile; and
(c) a computer-readable medium programmed with algorithmic logic to interpret the generated fluorescent signals from the eight discrete reaction pools and automatically classify the detected HPV targets into predefined clinical risk categories.
